(19) United States
(12) Patent Application Publication
(10) Pub. No.: US 2002/0177167 A1
(43) Pub. Date: Nov. 28, 2002

(54) Method and system for planning, performing, and assessing high-throughput screening of multicomponent chemical compositions and solid forms of compounds

(76) Inventors: Douglas A. Levinson, Sherborn, MA (US); Donovan Chin, Lexington, MA (US)

Correspondence Address: PENNIE AND EDMONDS, 1155 AVENUE OF THE AMERICAS, NEW YORK, NY, 100362711

(21) Appl. No.: 10/103,983

(22) Filed: Mar. 22, 2002

Related U.S. Application Data
(60) Provisional application No. 60/278,401, filed on Mar. 23, 2001.

(51) Int. Cl. G01N 33/53; G06F 19/00; G01N 33/48; G01N 33/50; G01N 33/543

(52) U.S. Cl. 435/7.1; 436/518; 702/19

(57) ABSTRACT

The present application is directed to the use of computerized data processing to plan, perform, and assess the results of high-throughput screening of multicomponent chemical compositions and solid forms of compounds. Systems utilized include databases of molecular descriptors and related compounds and their properties as determined empirically and through simulation, along with multidimensional visualization tools. Methods include methods for determining chemical compositions by performing steps including selecting a plurality of combinations of values of experimental parameters that can be varied by an automated experiment apparatus, determining a set of experimental results, and determining a second plurality of combinations of values based on the set of experimental results. Additional methods include selecting values of parameters that produce a composition, the values being relatively far from areas of rapid change or boundaries between solid forms.

### CROSS REFERENCE TO RELATED APPLICATIONS

This application is related to the U.S. provisional application No. 60/278,401 by Douglas A. Levinson and Donovan Chin, filed on Mar. 23, 2001, and entitled “METHOD AND SYSTEM FOR PLANNING, PERFORMING, AND ASSESSING HIGH-THROUGHPUT SCREENING OF MULTICOMPONENT CHEMICAL COMPOSITIONS AND SOLID FORMS OF COMPOUNDS,” which is incorporated herein by reference in its entirety.

### FIELD OF THE INVENTION

The present invention relates to the field of computerized data processing of experimental data.

### BACKGROUND OF THE INVENTION

Most chemical products embody compromises. In pharmaceuticals, for example, there are typically trade-offs between drug solubility, stability, absorption and bioavailability. FLUOXETINE, PROZAC's active agent, suffers from very low solubility in water and undergoes extensive first hepatic pass. LORATADINE, CLARITIN's active agent, is insoluble in water and also undergoes extensive first pass metabolism in the liver. PACLITAXEL, TAXOL's active agent, suffers from poor absorption due to its low water solubility.

Often these trade-offs can be usefully manipulated through changes of the solid form and/or the chemical formulation in which an active agent is delivered. The solubility, bioavailability, shelf-life, usability, taste and many other properties of the chemical product may vary in a complex way with the formulation due to interactions among the active agent and the excipients that make up the chemical product, and the particular use or administration method, thereof. Similarly, properties of the solid form of an ingredient, such as its crystal habit and morphology can significantly affect properties such as stability, bioavailability, and industrial processing. Selection of optimal formulations and solid form can therefore significantly alter the performance of pharmaceuticals and other chemical products. Dietary supplements, alternative medicines, nutraceuticals, sensory compounds, agrochemicals, and consumer and industrial formulations, also can benefit from reformulation and new solid forms.

The task of determining an optimal or near-optimal formulation is enormous. On the one hand, a property often can be optimized only at the expense of other desirable properties, so that no single property may be optimized in isolation. On the other, the properties of compounds or mixtures vary in a complex or unpredictable way with formulation parameters. Also, the types and ranges of formulation parameters that may be varied in manufacturing are very large.

More than 3,000 excipients are currently accepted and available for designing pharmaceutical compositions. A search for an optimum combination of excipients and active agents for even a relatively simple pharmaceutical composition is not trivial. Not only does one need to determine which of those excipients would be compatible with the active agent, but one has to determine the optimum values for such parameters as pH and relative concentrations of the components.

The problem grows geometrically with the number of excipients and other parameters considered. For example, simply to select a combination of two compounds out of a group of three hundred, without considering other variables such as relative concentrations, requires sifting through 44,850 combinations. This increases rapidly to 4,455,100 combinations for three compounds, and 330,791,175 combinations in the case of a four-compound mixture. Similar problems confront an effort to develop new solid forms of known substances.

In addition, because the conditions under which a formulation or solid form is manufactured, stored, administered or used typically vary over a significant range, the commercial usefulness of a formulation or solid form depend on the properties of the formulation or solid form over the expected range of conditions under which it will be manufactured, stored, administered or used. If the properties of the formulation change significantly over the expected range, or if the solid form is unstable or another solid form is produced at different points of the expected range, the usefulness of the formulation or solid form suffers. Selection of a commercially useful formulation or solid form therefore benefits from consideration of the behavior of the formulation or solid form over the expected range.

The scale of these problems may be reduced if relationships between one or more properties to be optimized and one or more molecular descriptors are discovered. A molecular descriptor as used herein is an empirical or theoretical datum that may be used in a quantitative structure-activity or structure-property relationship to predict molecular properties in complex environments. For a discussion of molecular descriptors, see Karelson, Molecular Descriptors in QSAR/QSPR, John Wiley & Sons, Inc. (2000), which is incorporated herein by reference. Many categories of compounds, such as pharmaceutical excipients, have been characterized based on a large number of molecular descriptors. Commercial and noncommercial databases of such characterizations are often available. Typically the molecular descriptors relevant to a desired property or properties are a small fraction of those that are measurable, calculable, or known. Moreover, the relationship between the relevant molecular descriptors and the desired property or properties often cannot be easily determined.

The magnitude of the problem does not arise solely from the extremely large number of possible combinations of relevant parameters that may be varied in manufacturing or experimentation. In many situations, neither the experimentally variable parameters nor the measurable or calculable characteristics of a compound or mixture of interest will have any known correlation with the property or properties which the experimentalist seeks to optimize. In the past, attempts have been made to characterize a material by performing one experiment at a time using a preselected combination of molecular descriptors and/or one or more bulk properties. This method of characterization is very time-consuming.

Recent advances in automation of experiments or experimental procedures have made it possible to perform tens or even hundreds of thousands of experiments in a relatively short period. Nevertheless, because the number and range of experimental parameters available to the experimentalist are extremely large, even hundreds of thousands of data points may be a very small fraction of accessible experiments that may be relevant to the properties of interest. Also, because the measured results may vary in a highly nonlinear fashion with the experimental parameters, unsophisticated selection of even a large number of data points may not accurately characterize the relationship between measured properties and experimental parameters. Thus, one may be able to collect hundreds of thousands of experimental data points and still fail to determine useful correlations or relationships between experimental or manufacturing parameters and desired properties. The range of possible experiments is simply too large for random or uniform sampling alone to yield optimal or near-optimal results.

There is thus a need to systematically integrate all available information in a manner that permits the useful deployment of a limited number of experiments to increase or maximize the probability of yielding compounds, compositions, or formulations that possess a desired property or set of properties over an expected range of conditions of manufacture, storage, administration or use, or combinations thereof.

### SUMMARY OF THE INVENTION

The present invention is directed to the computerized processing of data corresponding to experimental conditions and results for massively parallel experiments.

More specifically, in one aspect the present invention comprises a method for determining a multicomponent chemical composition, comprising the steps of selecting a combination of experimental parameters that may be varied by a high-throughput automated experimentation apparatus; determining a first plurality of distinct combinations of values of the experimental parameters, each combination corresponding to a distinct experiment; causing the automated experimentation apparatus to conduct a first set of experiments, each experiment of the first set corresponding to a distinct combination of values of the first plurality; determining a first collection of experimental results of the first set of experiments, the first collection comprising a plurality of individual result sets, each individual result set corresponding to a distinct experiment; based on the first collection of experimental results, determining a second plurality of distinct combinations of values of the experimental parameters, each combination corresponding to a distinct experiment; causing the automated experimentation apparatus to conduct a second set of experiments, each experiment of the second set corresponding to a distinct combination of values of the second plurality; determining a second collection of experimental results of the second set of experiments, the second collection comprising a plurality of individual result sets, each individual result set corresponding to a distinct experiment; selecting the multicomponent chemical composition of matter based on the first collection of experimental results and the second collection of experimental results.

In another aspect, the invention comprises a method for determining a solid form of a compound, comprising the steps of selecting a combination of experimental parameters that may be varied by a high-throughput automated experimentation apparatus; determining a first plurality of distinct combinations of values of the experimental parameters, each combination corresponding to a distinct experiment; causing the automated experimentation apparatus to conduct a first set of experiments, each experiment of the first set corresponding to a distinct combination of values of the first plurality; determining a first collection of experimental results of the first set of experiments, the first collection comprising a plurality of individual result sets, each individual result set corresponding to a distinct experiment; based on the first collection of experimental results, determining a second plurality of distinct combinations of values of the experimental parameters, each combination corresponding to a distinct experiment; causing the automated experimentation apparatus to conduct a second set of experiments, each experiment of the second set corresponding to a distinct combination of values of the second plurality; determining a second collection of experimental results of the second set of experiments, the second collection comprising a plurality of individual result sets, each individual result set corresponding to a distinct experiment; selecting the multicomponent chemical composition of matter based on the first collection of experimental results and the second collection of experimental results.

In another aspect, the invention comprises a method of estimating a property of a multicomponent chemical composition comprising the steps of receiving signals representing an experimental result set for each of a plurality of experiments conducted by a high-throughput automated experimentation apparatus; for each of at least a portion of the experiments, generating a predictive model based on signals characterizing each experimental result set according to the property to be estimated and signals characterizing the experiment with respect to a set of molecular descriptors; estimating the property for the multicomponent chemical composition by providing signals characterizing the multicomponent chemical composition with respect to the molecular descriptors, as input into the predictive model.

In still another aspect, the present invention comprises a method of estimating a property of a solid form of a compound, comprising the steps of receiving signals representing an experimental result set for each of a plurality of experiments conducted by a high-throughput automated experimentation apparatus; for each of at least a portion of the experiments, generating a predictive model based on signals characterizing each experimental result set according to the property to be estimated and signals characterizing the experiment with respect to a set of molecular descriptors; estimating the property for the solid form of a compound by providing signals characterizing the solid form of the compound with respect to the molecular descriptors as input into the predictive model.

In another aspect, the present invention comprises a method of estimating a property of a multicomponent chemical composition comprising the steps of receiving signals representing a simulation result set for each of a plurality of simulations of a multicomponent chemical composition; for at least a portion of the simulations, generating a predictive model based on signals characterizing each simulation result set according to the property to be estimated and signals characterizing the simulation results sets with respect to a set of molecular descriptors; receiving signals representing an experimental result set for each of a plurality of experiments conducted by a high-throughput automated experimentation apparatus; estimating the property for the multicomponent chemical composition by providing signals characterizing the multicomponent chemical composition with respect to the set of molecular descriptors as input to the predictive model.

In yet another aspect, the invention comprises a method of estimating a property of a solid form of a compound comprising the steps of receiving signals representing a simulation result set for each of a plurality of simulations of a solid form of a compound; for at least a portion of the simulations, training and generating a predictive model based on signals characterizing each simulation result set according to the property to be estimated and signals characterizing the simulation results sets with respect to a set of molecular descriptors; receiving signals representing an experimental result set for each of a plurality of experiments conducted by a high-throughput automated experimentation apparatus; estimating the property for the solid form of the compound by providing signals characterizing the solid form of the compound with respect to the set of molecular descriptors as input to the predictive model.

In another aspect, the present invention comprises a method of determining a multicomponent chemical composition comprising: (1) conducting a plurality of experiments a using high-throughput automated experimentation apparatus; (2) for each experiment, electronically storing data representing: (a) a set of experimental parameters, (b) a set of experimental results, and (c) a set of molecular descriptors characterizing an aspect of the experiment; (3) associating data from the plurality of experiments with previously stored data by querying a database comprising information not derived from the plurality of experiments; (4) processing data from the plurality of experiments with a processor programmed to apply a discriminator algorithm to associate at least one experiment with at least one classification.

In another aspect, the invention comprises a method of determining a solid form of a compound comprising: (1) conducting a plurality of experiments using a high-throughput automated experimentation apparatus; (2) for each experiment, electronically storing: (a) a set of experimental parameters, (b) a set of experimental results, (c) a set of molecular descriptors characterizing an aspect of the experiment; (3) associating data from the plurality of experiments with previously stored data by querying a database comprising information not derived from the plurality of experiments; (4) processing at least a portion of the experiment data and the associated previously stored data with a processor programmed to apply a discriminator algorithm to associate at least one experiment with at least one classification.

In yet another aspect, the invention comprises: a system for determining a multicomponent chemical composition comprising: (1) a database comprising at least one table, the at least one table further comprising: (a) a plurality of molecular descriptors, (b) a plurality of compound identifiers, (c) a plurality of compound/descriptor relations associating compound identifiers with molecular descriptors, (d) a plurality of empirically determined physical, chemical and biological parameters, (e) a plurality of compound/parameter relations associating compound identifiers with the empirically determined physical, chemical and biological parameters, (f) data representing results from a plurality of experiments performed with a high-throughput automated experimentation apparatus; (2) a query system for selecting subsets of related information from the at least one table; (3) a multidimensional representation generation module capable of generating visual representations of data sets having at least four dimensions; (4) a plurality of modeling modules, each module capable of receiving information selected by the query system and estimating at least one property of a multicomponent chemical composition.

In another aspect, the invention comprises a system for determining a solid form of a compound comprising: (1) a database comprising at least one table, the at least one table further comprising: (a) a plurality of molecular descriptors, (b) a plurality of compound identifiers, (c) a plurality of compound/descriptor relations associating compound identifiers with molecular descriptors, (d) a plurality of empirically determined physical, chemical and biological parameters, (e) a plurality of compound/parameter relations associating compound identifiers with the empirically determined physical, chemical and biological parameters, (f) data representing results from a plurality of experiments performed with a high-throughput automated experimentation apparatus; (2) a query system for selecting subsets of related information from the at least one table; (3) a multidimensional representation generation module capable of generating visual representations of data sets having at least four dimensions; (4) a plurality of modeling modules, each module capable of receiving information selected by the query system and estimating at least one property of a formulation.

In another aspect, the invention comprises a method for producing crystals comprising electronically calculating a set of predicted crystal polymorphs of a target compound; electronically calculating expected experimental results for the predicted crystal polymorphs; conducting a first plurality of crystallization experiments using a high-throughput automated experimentation apparatus; electronically comparing the expected experimental results with the actual experimental results to determine which predicted crystal polymorphs were produced.

In another aspect, the invention comprises a method for preparing a crystal form of a compound comprising: (1) performing simulated hydrogen-bond-biased simulated annealing to predict a plurality of crystal polymorphs of a target compound; (2) calculating expected properties of the predicted crystal polymorphs; (3) conducting a plurality of crystallization experiments using a high-throughput automated experimentation apparatus; (4) comparing measured properties of crystal forms produced by the plurality of crystallization experiments with the expected properties of the predicted crystal polymorphs to determine which predicted crystal polymorphs were produced by the experiments; (5) generating a predictive model of the relationship between experimental parameters and the crystal polymorphs produced; (6) calculating a set of experimental parameters for a second set of crystallization experiments from the predictive model; (7) optionally repeating steps 3-6 until a set of crystal polymorphs are obtained.

### BRIEF DESCRIPTION OF THE DRAWINGS

FIG. 1 is a schematic illustration of one example preferred embodiment.

FIG. 2 is an illustration of a display of a high-dimensional visualization in which the experimental results are represented as points of varying size, in a representation of a projection of a multidimensional space.

FIG. 3 is an illustration of an identification of certain groups of experimental results as exhibiting measured results of interest.

FIG. 4 is an illustration of additional data points corresponding to distinct experiments to more accurately characterize a formulation near results of interest.

FIG. 5 schematically illustrates a preferred method to assess a first collection of experimental results in a search for novel or known solid forms.

FIG. 6 schematically illustrates am architecture of a preferred example embodiment.

FIG. 7 is an illustration of a display of a high-dimensional visualization in which the experimental results are appear along the length of the axes, with each experiment appearing at the same place along all four axes and the width of each line on each axis is proportional to the normalized magnitude of the value represented by the axis for the corresponding experiment.

### DETAILED DESCRIPTION OF THE INVENTION

The present invention provides a system and associated methods for chemical knowledge acquisition through data acquisition, retrieval, and mining technologies.

Substances, such as pharmaceutical compounds can assume many different crystal forms and sizes. Particular emphasis has been put on these crystal characteristics in the pharmaceutical industry—especially polymorphic form, crystal size, crystal habit, and crystal-size distribution—since crystal structure and size can affect manufacturing, formulation, and pharmacokinetics, including bioavailability. There are four broad classes by which crystals of a given compound may differ: composition, habit, polymorphic form, and crystal size.

As used herein, composition refers to whether the solid-form is a single compound or a mixture of compounds. For example, solid-forms can be present in their free form, e.g., the free base of a compound having a basic nitrogen or as a salt, e.g., the hydrochloride salt of a basic nitrogen-containing compound. Composition also refers to crystals containing adduct molecules. During crystallization or precipitation an adduct molecule (e.g., a solvent or water) can be incorporated into the matrix, adsorbed on the surface, or trapped within the particle or crystal. Such compositions are referred to as inclusions, such as hydrates (water molecule incorporated in the matrix) and solvates (solvent trapped within a matrix). Whether a crystal forms as an inclusion can have a profound effect on the properties, such as the bioavailability or ease of processing or manufacture of a pharmaceutical. For example, inclusions may dissolve more or less readily or have different mechanical properties or strength than the corresponding non-inclusion compounds. A crystal habit refers to the various external shapes that a crystal assumes upon crystallization, which depend on, among others, the composition of the crystallizing medium. Those shapes may be cubic, tetragonal, orthorhombic, monoclinic, triclinic, rhomboidal, or hexagonal. Such information is important because the crystal habit has a large influence on the crystal's surface-to-volume ratio. Although crystal habits have the same internal structure and thus have identical single crystal- and powder-diffraction patterns, they can still exhibit different pharmaceutical properties (Haleblian 1975, J. Pharm. Sci., 64:1269). Thus means for discovering conditions or formulations that affect crystal habit are needed.

Polymorphism refers to the phenomenon in which a compound crystallizes into more than one distinct crystalline species (i.e., having a different internal structure) or shift from one crystalline species to another. The distinct species, which are known as polymorphs, can exhibit different optical properties, melting points, solubilities, chemical reactivities, dissolution rates, and different bioavailabilities. It is well known that different polymorphs of the same pharmaceutical can have different pharmacokinetics, for example, one polymorph can be absorbed more readily than its counterpart. In the extreme, only one polymorphic form of a given pharmaceutical may be suitable for disease treatment. Thus, the discovery and development of novel or beneficial polymorphs is extremely important, especially in the pharmaceutical area.

Amorphous solids, on the other hand, have no defined crystal shape and cannot be characterized according to habit or polymorphic form. An amorphous solid is in a high-energy structural state relative to its crystalline form which gives rise to instability problems.

It may crystallize during storage or shipping or an amorphous solid may be more sensitive to oxidation (Pikal et al., 1997, J. Pharm. Sci. 66:1312). A common amorphous solid is glass in which the atoms and molecules exist in a nonuniform array. Amorphous solids are usually the result of rapid solidification and can be conveniently identified (but not characterized) by x-ray powder diffraction, since these solids give very diffuse lines or no crystal diffraction pattern.

Crystals are normally obtained by dissolving a compound in a suitable solvent and then adjusting the conditions to induce crystal growth. The crystallization process commonly involves dissolving the compound at a temperature higher than its crystallization temperature. Upon cooling, at or below the compound's crystallization temperature, the solution becomes supersaturated which leads to the appearance of the crystals. Sometimes, crystal formation is induced by mechanically disturbing the solution, such as by scratching the inner surface of the solution container, or by seeding the solution with dust or crystals of the same compound. The pH, rate of cooling, type of solvent, solute-solvent ratio, additives such as surfactants, and inhibitors not only affect the purity of the crystals that form, but they may affect the crystal habit or polymorph that predominates. Other methods of crystal cultivation are sublimation, solvent evaporation, vapor diffusion, heating, crystallization from the melt, rapid pH change, thermal desolvation of crystalline solvates, and crystallization in the presence of additives (Guillory, Polymorphism in Pharmaceutical Solids, 186, 1999). Because of the extremely large number of possible combinations of components and experimental conditions, the range of conditions that may produce novel or known solid forms is very large, and locating optimal solid forms is commensurately difficult. As used herein, the term “array” means a plurality of samples, preferably, at least 24 samples each sample comprising a compound-of-interest and at least one component, wherein: (a) an amount of the compound-of-interest in each sample is less than about 100 micrograms; and (b) at least one of the samples comprises a solid-form of the compound-ofinterest. An array can comprise 2 or more samples, for example, 24, 36, 48, 96, or more samples, preferably 1000 or more samples, more preferably, 10,000 or more samples. An array can comprise one or more groups of samples also known as sub-arrays. For example, a group can be a 96-tube plate of sample tubes or a 96-well plate of sample wells in an array consisting of 100 or more plates. Each sample or selected samples or each sample group of selected sample groups in the array can be subjected to the same or different processing parameters; each sample or sample group can have different components or concentrations of components; or both to induce, inhibit, prevent, or reverse formation of solid-forms of the compound-of-interest. Arrays can be prepared by preparing a plurality of samples, each sample comprising a compound-of-interest and one or more components, then processing the samples to induce, inhibit, prevent, or reverse formation of solid-forms of the compound-of-interest.

As used herein, the term “sample” means a mixture of a compound-of-interest and one or more additional components to be subjected to various processing parameters and then screened to detect the presence or absence of solid-forms, preferably, to detect desired solid-forms with new or enhanced properties. In addition to the compound-of-interest, the sample comprises one or more components, preferably, 2 or more components, more preferably, 3 or more components. In general, a sample will comprise one compound-of-interest but can comprise multiple compounds-of-interest. Typically, a sample comprises less than about 1 g of the compound-of-interest, preferably, less than about 100 mg, more preferably, less than about 25 mg, even more preferably, less than about 1 mg, still more preferably less than about 100 micrograms, and optimally less than about 100 nanograms of the compound-of-interest. Preferably, the sample has a total volume of 100-250 ul.

As used herein, the term “pharmaceutical” means any substance that has a therapeutic, disease preventive, diagnostic, or prophylactic effect when administered to an animal or a human. The term pharmaceutical includes prescription pharmaceuticals and over the counter pharmaceuticals. Pharmaceuticals suitable for use in the invention include all those known or to be developed. A pharmaceutical can be a large molecule (i.e., molecules having a molecular weight of greater than about 1000 g/mol), such as oligonucleotides, polynucleotides, oligonucleotide conjugates, polynucleotide conjugates, proteins, peptides, peptidomimetics, or polysaccharides or small molecules (i.e., molecules having a molecular weight of less than about 1000 g/mol), such as hormones, steroids, nucleotides, nucleosides, or aminoacids.

Examples of suitable small molecule pharmaceuticals include, but are not limited to, cardiovascular pharmaceuticals, such as amlodipine, losartan, irbesartan, diltiazem, clopidogrel, digoxin, abciximab, furosemide, amiodarone, beraprost, tocopheryl; anti30 infective components, such as amoxicillin, clavulanate, azithromycin, itraconazole, acyclovir, fluconazole, terbinafine, erythromycin, and acetyl sulfisoxazole; psychotherapeutic components, such as sertraline, venlafaxine, bupropion, olanzapine, buspirone, alprazolam, methylphenidate, fluvoxamine, and ergoloid; gastrointestinal products, such as lansoprazole, ranitidine, famotidine, ondansetron, granisetron, sulfasalazine, and infliximab; respiratory therapies, such as loratadine, fexofenadine, cetirizine, fluticasone, salmeterol, and budesonide; cholesterol reducers, such as atorvastatin calcium, lovastatin, bezafibrate, ciprofibrate, and gemfibrozil; cancer and cancer-related therapies, such as paclitaxel, carboplatin, tamoxifen, docetaxel, epirubicin, leuprolide, bicalutamide, goserelin implant, irinotecan, gemcitabine, and sargramostim; blood modifiers, such as epoetin alfa, enoxaparin sodium, and antihemophilic factor; antiarthritic components, such as celecoxib, nabumetone, misoprostol, and rofecoxib; AIDS and AIDS-related pharmaceuticals, such as lamivudine, indinavir, stavudine, and lamivudine; diabetes and diabetes-related therapies, such as metformin, troglitazone, and acarbose; biologicals, such as hepatitis B vaccine, and hepatitis A vaccine; hormones, such as estradiol, mycophenolate mofetil, and methylprednisolone; analgesics, such as tramadol hydrochloride, fentanyl, metamizole, ketoprofen, morphine, lysine acetylsalicylate, ketoralac tromethamine, loxoprofen, and ibuprofen; dermatological products, such as isotretinoin and clindamycin; anesthetics, such as propofol, midazolam, and lidocaine hydrochloride; migraine therapies, such as sumatriptan, zolmitriptan, and rizatriptan; sedatives and hypnotics, such as zolpidem, zolpidem, triazolam, and hycosine butylbromide; imaging components, such as iohexol, technetium, TC99M, sestamibi, iomeprol, gadodiamide, ioversol, and iopromide; and diagnostic and contrast components, such as alsactide, americium, betazole, histamine, mannitol, metyrapone, petagastrin, phentolamine, radioactive B₁₂, gadodiamide, gadopentetic acid, gadoteridol, and perflubron. Other pharmaceuticals for use in the invention include those listed in Table 1 below, which suffer from problems that could be mitigated by developing new administration formulations according to the arrays and methods of the invention.

Other examples of suitable pharmaceuticals are listed in 2000 Med Ad News 19:5660 and The Physicians Desk Reference, 53rd edition, 792-796, Medical Economics Company (1999), both of which are incorporated herein by reference.

Examples of suitable veterinary pharmaceuticals include, but are not limited to, vaccines, antibiotics, growth enhancing components, and dewormers. Other examples of suitable veterinary pharmaceuticals are listed in The Merck Veterinary Manual, 8th ed., Merck and Co., Inc., Rahway, N.J., 1998; (1997) The Encyclopedia of Chemical Technology, 24 Kirk-Othmer (4^{th} ed. at 826); and Veterinary Drugs in ECT 2nd ed., Vol 21, by A. L. Shore and R. J. Magee, American Cyanamid Co.

As used herein, the term “dietary supplement” means a non-caloric or insignificant-caloric substance administered to an animal or a human to provide a nutritional benefit or a non-caloric or insignificant-caloric substance administered in a food to impart the food with an aesthetic, textural, stabilizing, or nutritional benefit. Dietary supplements include, but are not limited to, fat binders, such as caducean; fish oils; plant extracts, such as garlic and pepper extracts; vitamins and minerals; food additives, such as preservatives, acidulents, anticaking components, antifoaming components, antioxidants, bulking components, coloring components, curing components, dietary fibers, emulsifiers, enzymes, firming components, humectants, leavening components, lubricants, non-nutritive sweeteners, food-grade solvents, thickeners; fat substitutes, and flavor enhancers; and dietary aids, such as appetite suppressants.

Examples of suitable dietary supplements are listed in (1994) The Encyclopedia of Chemical Technology, 11 Kirk-Othmer (4^{th} ed. at 805-833). Examples of suitable vitamins are listed in (1998) The Encyclopedia of Chemical Technology, 25 Kirk-Othmer (4^{th} ed. at 1) and Goodman & Gilman's: The Pharmacological Basis of Therapeutics, 9th Edition, eds. Joel G. Harman and Lee E. Limbird, McGraw-Hill, 1996 p. 1547, both of which are incorporated by reference herein. Examples of suitable minerals are listed in The Encyclopedia of Chemical Technology, 16 Kirk-Othmer (4^{th} ed. at 746) and “Mineral Nutrients” in ECT 3rd ed., Vol 15, pp. 570-603, by C. L. Rollinson and M. G. Enig, University of Maryland, both of which are incorporated herein by reference.

As used herein, the term “alternative medicine” means a substance, preferably a natural substance, such as a herb or an herb extract or concentrate, administered to a subject or a patient for the treatment of disease or for general health or well being, wherein the substance does not require approval by the FDA. Examples of suitable alternative medicines include, but are not limited to, ginkgo biloba, ginseng root, valerian root, oak bark, kava kava, echinacea, harpagophyti radix, others are listed in The Complete German Commission E Monographs: Therapeutic Guide to Herbal Medicine, Mark Blumenthal et al. eds., Integrative Medicine Communications 1998, incorporated by reference herein.

As used herein the term “nutraceutical” means a food or food product having both caloric value and pharmaceutical or therapeutic properties. Example of nutraceuticals include garlic, pepper, brans and fibers, and health drinks Examples of suitable Nutraceuticals are listed in M. C. Linder, ed. Nutritional Biochemistry and Metabolism with Clinical Applications, Elsevier, New York, 1985; Pszczola et al., 1998 Food technology 52:30-37 and Shukla et al., 1992 Cereal Foods World 37:665-666.

As used herein, the term “sensory-material” means any chemical or substance, known or to be developed, that is used to provide an olfactory or taste effect in a human or an animal, preferably, a fragrance material, a flavor material, or a spice. A sensory-material also includes any chemical or substance used to mask an odor or taste. Examples of suitable fragrances materials include, but are not limited to, musk materials, such as civetone, ambrettolide, ethylene brassylate, musk xylene, Tonalide®, and Glaxolide®; amber materials, such as ambrox, ambreinolide, and ambrinol; sandalwood materials, such as α-santalol, β-santalol, Sandalore®, and Bacdanol®; patchouli and woody materials, such as patchouli oil, patchouli alcohol, Timberol® and Polywood®; materials with floral odors, such as Givescone®, damascone, irones, linalool, Lilial®, Lilestralis®, and dihydrojasmonate. Other examples of suitable fragrance materials for use in the invention are listed in Perfumes: Art, Science, Technology, P. M. Muller ed. Elsevier, New York, 1991, incorporated herein by reference. Examples of suitable flavor materials include, but are not limited to, benzaldehyde, anethole, dimethyl sulfide, vanillin, methyl anthranilate, nootkatone, and cinnamyl acetate. Examples of suitable spices include but are not limited to allspice, tarrogon, clove, pepper, sage, thyme, and coriander. Other examples of suitable flavor materials and spices are listed in Flavor and Fragrance Materials-1989, Allured Publishing Corp. Wheaton, Ill., 1989; Bauer and Garbe Common Flavor and Fragrance Materials, VCH Verlagsgesellschaft, Weinheim, 1985; and (1994) The Encyclopedia of Chemical Technology, 11 Kirk-Othmer (4^{th} ed. at 1-61), all of which are incorporated by reference herein.

As used herein, the term “agrochemical” means any substance known or to be developed that is used on the farm, yard, or in the house or living area to benefit gardens, crops, ornamental plants, shrubs, or vegetables or kill insects, plants, or fungi. Examples of suitable agrochemicals for use in the invention include pesticides, herbicides, fungicides, insect repellants, fertilizers, and growth enhancers. For a discussion of agrochemicals see The Agrochemicals Handbook (1987) 2nd Edition, Hartley and Kidd, editors: The Royal Society of Chemistry, Nottingham, England.

Pesticides include chemicals, compounds, and substances administered to kill vermin such as bugs, mice, and rats and to repel garden pests such as deer and woodchucks. Examples of suitable pesticides that can be used according to the invention include, but are not limited to, abamectin (acaricide), bifenthrin (acaricide), cyphenothrin (insecticide), imidacloprid (insecticide), and prallethrin (insectide). Other examples of suitable pesticides for use in the invention are listed in Crop Protection Chemicals Reference, 6th ed., Chemical and Pharmaceutical Press, John Wiley & Sons Inc., New York, 1990; (1996) The Encyclopedia of Chemical Technology, 18 Kirk-Othmer (4^{th} ed. at 311-341); and Hayes et al, Handbook of Pesticide Toxicology, Academic Press, Inc., San Diego, Calif., 1990, all of which are incorporated by reference herein.

Herbicides include selective and non-selective chemicals, compounds, and substances administered to kill plants or inhibit plant growth. Examples of suitable herbicides include, but are not limited to, photosystem I inhibitors, such as actifluorfen; photosystem II inhibitors, such as atrazine; bleaching herbicides, such as fluridone and difunon; chlorophyll biosynthesis inhibitors, such as DTP, clethodim, sethoxydim, methyl haloxyfop, tralkoxydim, and alacholor; inducers of damage to antioxidative system, such as paraquat; amino-acid and nucleotide biosynthesis inhibitors, such as phaseolotoxin and imazapyr; cell division inhibitors, such as pronamide; and plant growth regulator synthesis and function inhibitors, such as dicamba, chloramben, dichlofop, and ancymidol. Other examples of suitable herbicides are listed in Herbicide Handbook, 6th ed., Weed Science Society of America, Champaign, Ill. 1989; (1995) The Encyclopedia of Chemical Technology, 13 Kirk-Othmer (4^{th} ed. at 73-136); and Duke, Handbook of Biologically Active Phytochemicals and Their Activities, CRC Press, Boca Raton, Fla., 1992, all of which are incorporated herein by reference.

Fungicides include chemicals, compounds, and substances administered to plants and crops that selectively or non-selectively kill fungi. For use in the invention, a fungicide can be systemic or non-systemic. Examples of suitable non-systemic fungicides include, but are not limited to, thiocarbamate and thiurame derivatives, such as ferbam, ziram, thiram, and nabam; imides, such as captan, folpet, captafol, and dichlofluanid; aromatic hydrocarbons, such as quintozene, dinocap, and chloroneb; dicarboximides, such as vinclozolin, chlozolinate, and iprodione. Example of systemic fungicides include, but are not limited to, mitochondiral respiration inhibitors, such as carboxin, oxycarboxin, flutolanil, fenfuram, mepronil, and methfuroxam; microtubulin polymerization inhibitors, such as thiabendazole, fuberidazole, carbendazim, and benomyl; inhibitors of sterol biosynthesis, such as triforine, fenarimol, nuarimol, imazalil, triadimefon, propiconazole, flusilazole, dodemorph, tridemorph, and fenpropidin; and RNA biosynthesis inhibitors, such as ethirimol and dimethirimol; phopholipic biosynthesis inhibitors, such as ediphenphos and iprobenphos. Other examples of suitable fungicides are listed in Torgeson, ed., Fungicides: An Advanced Treatise, Vols. 1 and 2, Academic Press, Inc., New York, 1967 and (1994) The Encyclopedia of Chemical Technology, 12 Kirk-Othmer (4^{th} ed. at 73-227), all of which are incorporated herein by reference.

As used herein, a “consumer formulation” means a formulation for consumer use, not intended to be absorbed or ingested into the body of a human or animal, comprising an active component. Preferably, it is the active component that is investigated as the compound-of-interest in the arrays and methods of the invention. Consumer formulations include, but are not limited to, cosmetics, such as lotions, facial makeup; antiperspirants and deodorants, shaving products, and nail care products; hair products, such as and shampoos, colorants, conditioners; hand and body soaps; paints; lubricants; adhesives; and detergents and cleaners.

As used herein an “industrial formulation” means a formulation for industrial use, not intended to be absorbed or ingested into the body of a human or animal, comprising an active component. Preferably, it is the active component of industrial formulation that is investigated as the compound-of-interest in the arrays and methods of the invention. Industrial formulations include, but are not limited to, polymers; rubbers; plastics; industrial chemicals, such as solvents, bleaching agents, inks, dyes, fire retardants, antifreezes and formulations for deicing roads, cars, trucks, jets, and airplanes; industrial lubricants; industrial adhesives; construction materials, such as cements.

One of skill in the art will readily be able to choose active and inactive components used in consumer and industrial formulations and set up arrays according to the invention. Such active components and inactive components are well known in the literature and the following references are provided merely by way of example. Active components and inactive components for use in cosmetic formulations are listed in (1993) The Encyclopedia of Chemical Technology, 7 Kirk-Othmer (4^{th} ed. at 572-619); M. G. de Navarre, The Chemistry and Manufacture of Cosmetics, D. Van Nostrand Company, Inc., New York, 1941; CTFA International Cosmetic Ingredient Dictionary and Handbook, 8th Ed., CTFA, Washington, D.C., 2000; and A. Nowak, Cosmetic Preparations, Micelle Press, London, 1991. All of which are incorporated by reference herein. Active components and inactive components for use in hair care products are listed in (1994) The Encyclopedia of Chemical Technology, 12 Kirk-Othmer (4^{th} ed. at 881-890) and Shampoos and Hair Preparations in ECT 1st ed., Vol. 12, pp. 221-243, by F. E. Wall, both of which are incorporated by reference herein. Active components and inactive components for use in hand and body soaps are listed in (1997) The Encyclopedia of Chemical Technology, 22 Kirk-Othmer (4^{th} ed. at 297-396), incorporated by reference herein. Active components and inactive components for use in paints are listed in (1996) The Encyclopedia of Chemical Technology, 17 Kirk-Othmer (4^{th} ed. at 1049-1069) and “Paint” in ECT 1st ed., Vol. 9, pp. 770-803, by H. E. Hillman, Eagle Paint and Varnish Corp, both of which are incorporated by reference herein. Active components and inactive components for use in consumer and industrial lubricants are listed in (1995) The Encyclopedia of Chemical Technology, 15 Kirk-Othmer (4^{th} ed. at 463-517); D. D. Fuller, Theory and practice of Lubrication for Engineers, 2nd ed., John Wiley & Sons, Inc., 1984; and A. Raimondi and A. Z. Szeri, in E. R. Booser, eds., Handbook of Lubrication, Vol. 2, CRC Press Inc., Boca Raton, Fla., 1983, all of which are incorporated by reference herein. Active components and inactive components for use in consumer and industrial adhesives are listed in (1991) The Encyclopedia of Chemical Technology, 1 Kirk-Othmer (4^{th} ed. at 445-465) and I. M. Skeist, ed. Handbook of Adhesives, 3rd ed. Van Nostrand-Reinhold, New York, 1990, both of which are incorporated herein by reference. Active components and inactive components for use in polymers are listed in (1996) The Encyclopedia of Chemical Technology, 19 Kirk-Othmer (4^{th} ed. at 881-904), incorporated herein by reference. Active components and inactive components for use in rubbers are listed in (1997) The Encyclopedia of Chemical Technology, 21 Kirk-Othmer (4^{th} ed. at 460-591), incorporated herein by reference. Active components and inactive components for use in plastics are listed in (1996) The Encyclopedia of Chemical Technology, 19 Kirk-Othmer (4^{th} ed. at 290-316), incorporated herein by reference. Active components and inactive components for use with industrial chemicals are listed in Ash et al., Handbook of Industrial Chemical Additives, VCH Publishers, New York 1991, incorporated herein by reference. Active components and inactive components for use in bleaching components are listed in (1992) The Encyclopedia of Chemical Technology, 4 Kirk-Othmer (4^{th} ed. at 271-311), incorporated herein by reference. Active components and inactive components for use inks are listed in (1995) The Encyclopedia of Chemical Technology, 14 Kirk-Othmer (4^{th} ed. at 482-503), incorporated herein by reference. Active components and inactive components for use in dyes are listed in (1993) The Encyclopedia of Chemical Technology, 8 Kirk-Othmer (4^{th} ed. at 533-860), incorporated herein by reference. Active components and inactive components for use in fire retardants are listed in (1993) The Encyclopedia of Chemical Technology, 10 Kirk-Othmer (4^{th} ed. at 930-1022), incorporated herein by reference. Active components and inactive components for use in antifreezes and deicers are listed in (1992) The Encyclopedia of Chemical Technology, 3 Kirk-Othmer (4^{th} ed. at 347-367), incorporated herein by reference. Active components and inactive components for use in cement are listed in (1993) The Encyclopedia of Chemical Technology, 5 Kirk-Othmer (4^{th} ed. at 564), incorporated herein by reference.

As used herein, the term “component” means any substance that is combined, mixed, or processed with the compound-of-interest to form a sample or impurities, for example, trace impurities left behind after synthesis or manufacture of the compound-of-interest. The term component includes solvents in the sample. The term component also encompasses the compound-of-interest itself. The compound-of-interest to be screened can be any useful solid compound including, but not limited to, pharmaceuticals, dietary supplements, nutraceuticals, agrochemicals, or alternative medicines. The invention is particularly well-suited for screening solid-forms of a single low-molecular-weight organic molecules. Thus, the invention encompasses arrays of diverse solid-forms of a single low-molecular-weight molecule.

A single substance can exist in one or more physical states having different properties thereby classified herein as different components. For instance, the amorphous and crystalline forms of an identical compound are classified as different components. Components can be large molecules (i.e., molecules having a molecular weight of greater than about 1000 g/mol), such as large-molecule pharmaceuticals, oligonucleotides, polynucleotides, oligonucleotide conjugates, polynucleotide conjugates, proteins, peptides, peptidomimetics, or polysaccharides or small molecules (i.e., molecules having a molecular weight of less than about 1000 g/mol) such as small-molecule pharmaceuticals, hormones, nucleotides, nucleosides, steroids, or aminoacids.

Components can also be chiral or optically-active substances or compounds, such as optically-active solvents, optically-active reagents, or optically-active catalysts. Preferably, components promote or inhibit or otherwise effect precipitation, formation, crystallization, or nucleation of solid-forms, preferably, solid-forms of the compound-of-interest. Thus, a component can be a substance whose intended effect in an array sample is to induce, inhibit, prevent, or reverse formation of solid-forms of the compound-of-interest. Examples of components include, but are not limited to, excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; additives that inhibit crystallization or solid formation; optically-active solvents; optically-active reagents; optically-active catalysts; and even packaging or processing reagents.

Components include acidic substances and basic substances. Such substances can react to form a salt with the compound-of-interest or other components present in a sample. When a salt of the compound-of-interest is desired, salt forming components will generally be used in stoichiometric quantities. Components that are basic in nature are capable of forming a wide variety of salts with various inorganic and organic acids. For example, suitable acids are those that form the following salts with basic compounds: chloride, bromide, iodide, acetate, salicylate, benzenesulfonate, benzoate, bicarbonate, bitartrate, calcium edetate, camsylate, carbonate, citrate, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)). Components that include an amino moiety also can form pharmaceutically-acceptable salts with various amino acids, in addition to the acids mentioned above.

The term “excipient” as used herein refers to substances used to formulate actives into pharmaceutical formulations. Preferably, an excipient does not lower or interfere with the primary therapeutic effect of the active, more preferably, an excipient is therapeutically inert. The term “excipient” encompasses carriers, solvents, diluents, vehicles, stabilizers, and binders. Excipients can also be those substances present in a pharmaceutical formulation as an indirect result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

Examples of suitable excipients include, but are not limited to, acidulents, such as lactic acid, hydrochloric acid, and tartaric acid; solubilizing components, such as non-ionic, cationic, and anionic surfactants; absorbents, such as bentonite, cellulose, and kaolin; alkalizing components, such as diethanolamine, potassium citrate, and sodium bicarbonate; anticaking components, such as calcium phosphate tribasic, magnesium trisilicate, and talc; antimicrobial components, such as benzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, hydroxyethyl cellulose; dextrin, gelatin, guar gum, magnesium aluminum silicate, maltodextrin, povidone, starch, vegetable oil, and zein; buffering components, such as sodium phosphate, malic acid, and potassium citrate; chelating components, such as EDTA, malic acid, and maltol; coating components, such as adjunct sugar, cetyl alcohol, polyvinyl alcohol, carnauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as collodial silicon dioxide, croscarmellose sodium, magnesium aluminum silicate, potassium polacrilin, and sodium starch glycolate; dispersing components, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying components, such as anionic emulsifying wax, monoethanolamine, and medium chain triglycerides; flavoring components, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitosterate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, for indigo carmine, polyethylene glycol, for sunset yellow, for tartazine, triacetin; stabilizing components, such as cyclodextrins, albumin, xanthan gum; and tonicity components, such as glycerol, dextrose, potassium chloride, and sodium chloride; and mixture thereof. Other examples of suitable excipients, such as binders and fillers are listed in Remington's Pharmaceutical Sciences, 18th Edition, ed. Alfonso Gennaro, Mack Publishing Co. Easton, Pa., 1995 and Handbook of Pharmaceutical Excipients, 3rd Edition, ed. Arthur H. Kibbe, American Pharmaceutical Association, Washington D.C. 2000, both of which are incorporated herein by reference.

In general, the arrays of the invention will contain a solvent as one on the components. Solvents may influence and direct the formation of solid-forms through polarity, viscosity, boiling point, volatility, charge distribution, and molecular shape. The solvent identity and concentration is one way to control saturation. Indeed, one can crystallize under isothermal conditions by simply adding a nonsolvent to an initially subsaturated solution. One can start with an array of a solution of the compound-of-interest in which varying amounts of nonsolvent are added to each of the individual elements of the array. The solubility of the compound is exceeded when some critical amount of nonsolvent is added. Further addition of the nonsolvent increases the supersaturation of the solution and, therefore, the growth rate of the crystals that are grown.

As used herein, the term “experimental parameters” means the physical or chemical conditions under which a sample is subjected and the time during which the sample is subjected to such conditions. Experimental parameters include, but are not limited to, the temperature, time, pH, amount or the concentration of a component, component identity, solvent removal rate, and solvent composition. Sub-arrays or even individual samples within an array can be subjected to processing parameters that are different from the processing parameters to which other sub-arrays or samples, within the same array, are subjected. Processing parameters will differ between sub-arrays or samples when they are intentionally varied to induce a measurable change in the sample's properties. Thus, according to the invention, minor variations, such as those introduced by slight adjustment errors, are not considered intentionally varied.

When referring to an interaction between components, an “interaction” means that the components as a mixture display a property (e.g., the ability to solubilize a specific pharmaceutical) of a different magnitude or value than the same property displayed by each component in isolation. Interactions between components will affect the properties of samples. Merely for example, a particular combination and ratio of excipients can interact such that the combination has a high solubilizing power for a particular pharmaceutical. Once such an interaction is detected, it can be exploited to develop enhanced formulations for the pharmaceutical.

As used herein, the term “property” means a structural, physical, pharmacological, or chemical characteristic of a sample, preferably, a structural, physical, pharmacological, or chemical characteristics of a compound-of-interest. The properties of a sample, as well as the interactions or the manifestations or outcomes of those interactions arising from or involving the original sample, can be analyzed using methods or techniques known in the art. Some examples of these methods or techniques are Raman and infrared spectroscopy, ultraviolet spectroscopy, second harmonic generation, x-ray diffraction, scanning electron microscopy, transmission electron microscopy, near field scanning optical microscopy, far field scanning optical microscopy, atomic force microscopy, micro-thermal analysis, differential analyis, nuclear magnetic resonance spectroscopy, gas chromatography, and high-pressure or high-performance liquid chromatography.

Preferred properties are those that relate to the efficacy, safety, stability, or utility of the compound-of-interest. For example, regarding pharmaceutical, dietary supplement, alternative medicine, and nutraceutical compounds and substances, properties include physical properties, such as stability, solubility, dissolution, permeability, and partitioning; mechanical properties, such as compressibility, compactability, and flow characteristics; the formulation's sensory properties, such as color, taste, and smell; and properties that affect the utility, such as absorption, bioavailability, toxicity, metabolic profile, and potency. Other properties include those which affect the compound-of-interest's behavior and ease of processing in a crystallizer or a formulating machine. For a discussion of industrial crystallizers and properties thereof see (1993) The Encyclopedia of Chemical Technology, 7 Kirk-Othmer (4^{th} ed. pp. 720-729). Such processing properties are closely related to the solid-form's mechanical properties and its physical state, especially degree of agglomeration. Concerning pharmaceuticals, dietary supplements, alternative medicines, and nutraceuticals, optimizing physical and utility properties of their solid-forms can result in a lowered required dose for the same therapeutic effect. Thus, there are potentially fewer side effects that can improve patient compliance.

Structural properties include, but are not limited to, whether the compound-of-interest is crystalline or amorphous, and if crystalline, the polymorphic form and a description of the crystal habit. Structural properties also include the composition, such as whether the solid-form is a hydrate, solvate, or a salt. Examples of structural property are surface-to-volume ratio and the degree of agglomeration of the particles. Surface-to-volume ratio decreases with the degree of agglomeration. It is well known that a high surface-to-volume ratio improves the solubility rate. Small-size particles have high surface-to-volume ratio. The surface-to-volume ratio is also influenced by the crystal habit, for example, the surface-to-volume ratio increases from spherical shape to needle shape to dendritic shape. Porosity also affects the surface-to-volume ratio, for example, solid-forms having channels or pores (e.g., inclusions, such as hydrates and solvates) have a high surface-to-volume ratio.

Still another structural property is particle size and particle-size distribution. For example, depending on concentrations, the presence of inhibitors or impurities, and other conditions, particles can form from solution in different sizes and size distributions. Particulate matter, produced by precipitation or crystallization, has a distribution of sizes that varies in a definite way throughout the size range. Particle- and crystal-size distribution is generally expressed as a population distribution relating to the number of particles at each size. In pharmaceuticals, particle and crystal size distribution have very important clinical aspects, such as bioavailability. Thus, compounds or compositions that promote small crystal size can be of clinical importance.

Physical properties include, but are not limited to, physical stability, melting point, solubility, strength, hardness, compressibility, and compactability. Physical stability refers to a compound's or composition's ability to maintain its physical form, for example maintaining particle size; maintaining crystal or amorphous form; maintaining complexed form, such as hydrates and solvates; resistance to absorption of ambient moisture; and maintaining of mechanical properties, such as compressibility and flow characteristics. Methods for measuring physical stability include spectroscopy, sieving or testing, microscopy, sedimentation, stream scanning, and light scattering. Polymorphic changes, for example, are usually detected by differential scanning calorimetry or quantitative infrared analysis. For a discussion of the theory and methods of measuring physical stability see Fiese et al., in The Theory and Practice of Industrial Pharmacy, 3rd ed., Lachman L.; Lieberman, H. A.; and Kanig, J. L. Eds., Lea and Febiger, Philadelphia, 1986 pp. 193-194 and Remington's Pharmaceutical Sciences, 18th Edition, ed. Alfonso Gennaro, Mack Publishing Co. Easton, Pa., 1995, pp. 1448-1451, both of which are incorporated herein by reference.

Solubility refers to the equilibrium solubility or steady state and is measured as weight component/volume solvent. When an active component, such as a pharmaceutical substance has an aqueous solubility of less than about 1 milligram/milliliter in the physiological pH range of 1-7, a potential bioavailability problem exists. Descriptive terms used to describe solubility given in parts of solvent for 1 part of solvent are: very soluble (<1 part); freely soluble (from 1 to 10 parts); soluble (from 10 to 30 parts); sparingly soluble (from 30 to 100 parts); slightly soluble (from 100 to 1,000 parts); very slightly soluble (from 1,000 to 10,000 parts); and insoluble (>10,000 parts). For a discussion of solution and phase equilibria see Remington's Pharmaceutical Sciences, 18th Edition, ed. Alfonso Gennaro, Mack Publishing Co. Easton, Pa., 1995, Ch. 16, incorporated herein by reference.

The solubility can be tested by mixing the sample with a test solvent and agitating the sample at a constant temperature until equilibrium is achieved. Equilibrium usually occurs upon agitating the samples for 6 to 24 hours. If the component is acidic or basic, its solubility can be influenced by pH and one of skill in the art will take such factors into consideration when testing the solubility properties of a sample. Once equilibrium has occurred, the sample can be tested to determine the amount of component dissolved using standard technology, such as mass spectroscopy, HPLC, UV spectroscopy, fluorescence spectroscopy, gas chromatography, optical density, or by colorimetery. For a discussion of the theory and methods of measuring solubility see Streng et al., 1984 J. Pharm. Sci. 63:605; Kaplan 1972 Drug Metab. Rev. 1:15; and Remington's Pharmaceutical Sciences, 18th Edition, ed. Alfonso Gennaro, Mack Publishing Co. Easton, Pa., 1995, pp.1456-1457, all three of which are incorporated herein by reference. For a discussion of heat of dissolution, pKa, and pH solubility profile effects and techniques for measurement thereof see Fiese et al., in The Theory and Practice of Industrial Pharmacy, 3rd ed., Lachman L.; Lieberman, H. A.; and Kanig, J. L. Eds., Lea and Febiger, Philadelphia, 1986 pp. 185-188, incorporated herein by reference.

Dissolution refers to the process by which a solid enters into solution. Several factors affect dissolution such as solubility, particle size, crystalline state, and the presence of diluents, disintegrants, or other excipients. For a discussion of the theory and methods of measuring dissolution see Remington's Pharmaceutical Sciences, 18th Edition, ed. Alfonso Gennaro, Mack Publishing Co. Easton, Pa., 1995, Chapter 34, incorporated herein by reference.

Chemical properties include, but are not limited to chemical stability, such as susceptibility to oxidation and reactivity with other compounds, such as acids, bases, or chelating agents. Chemical stability refers to resistance to chemical reactions induced, for example, by heat, ultraviolet radiation, moisture, chemical reactions between components, or oxygen. Well known methods for measuring chemical stability include mass spectroscopy, UV-VIS spectroscopy, HPLC, gas chromatography, and liquid chromatography-mass spectroscopy (LC-MS). For a discussion of the theory and methods of measuring chemical stability see Xu et al., Stability-Indicating HPLC Methods for Drug Analysis American Pharmaceutical Association, Washington D.C. 1999 and Remington's Pharmaceutical Sciences, 18th Edition, ed. Alfonso Gennaro, Mack Publishing Co. Easton, Pa., 1995, pp. 1458-1460, both of which are incorporated herein by reference.

As used herein, the term “solid-form” means a form of a solid substance, element, or chemical compound that is defined and differentiated from other solid-forms according to its physical state and properties.

The basic requirements for array and sample preparation and screening thereof are: (1) a distribution mechanism to add components and the compound-of-interest to separate sites, for example, on an array plate having sample wells or sample tubes. Preferably, the distribution mechanism is automated and controlled by computer software and can vary at least one addition variable, e.g., the identity of the component(s) and/or the component concentration, more preferably, two or more variables. Such material handling technologies and robotics are well known to those skilled in the art. If desired, individual components can be placed at the appropriate sample site manually. This pick and place technique is also known to those skilled in the art. (2) a screening mechanism to test each sample to detect a change in physical state or for one or more properties. Preferably, the testing mechanism is automated and driven by a computer. Preferably, the system further comprises a processing mechanism to process the samples after component addition. Optionally, the system can have a processing station the process the samples after preparation.

A number of companies have developed array systems that can be adapted for use in the invention disclosed herein. Such systems may require modification, which is well within ordinary skill in the art. Examples of companies having array systems include Gene Logic of Gaithersburg, Md. (see U.S. Pat. No. 5,843,767 to Beattie), Luminex Corp., Austin, Tex., Beckman Instruments, Fullerton, Calif., MicroFab Technologies, Plano, Tex., Nanogen, San Diego, Calif., and Hyseq, Sunnyvale, Calif. These devices test samples based on a variety of different systems. All include thousands of microscopic channels that direct components into test wells, where reactions can occur. These systems are connected to computers for analysis of the data using appropriate software and data sets. The Beckman Instruments system can deliver nanoliter samples of 96 or 384-arrays, and is particularly well suited for hybridization analysis of nucleotide molecule sequences. The MicroFab Technologies system delivers sample using inkjet printers to aliquot discrete samples into wells. These and other systems can be adapted as required for use herein. For example, the combinations of the compound-of-interest and various components at various concentrations and combinations can be generated using standard formulating software (e.g., Matlab software, commercially available from Mathworks, Natick, Mass.). The combinations thus generated can be downloaded into a spread sheet, such as Microsoft EXCEL. From the spread sheet, a work list can be generated for instructing the automated distribution mechanism to prepare an array of samples according to the various combinations generated by the formulating software.

The work list can be generated using standard programming methods according to the automated distribution mechanism that is being used. The use of so-called work lists simply allows a file to be used as the process command rather than discrete programmed steps. The work list combines the formulation output of the formulating program with the appropriate commands in a file format directly readable by the automatic distribution mechanism. The automated distribution mechanism delivers at least one compound-of-interest, such as a pharmaceutical, as well as various additional components, such as solvents and additives, to each sample well. Preferably, the automated distribution mechanism can deliver multiple amounts of each component. Automated liquid and solid distribution systems are well known and commercially available, such as the Tecan Genesis, from Tecan-US, RTP, North Carolina. The robotic arm can collect and dispense the solutions, solvents, additives, or compound-of-interest form the stock plate to a sample well or sample tube. The process is repeated until array is completed, for example, generating an array that moves from wells at left to right and from top to bottom in increasing polarity or non-polarity of solvent. The samples are then mixed. For example, the robotic arm moves up and down in each well plate for a set number of times to ensure proper mixing.

Liquid handling devices manufactured by vendors such as Tecan, Hamilton and Advanced Chemtech are all capable of being used in the invention. A prerequisite for all liquid handling devices is the ability to dispense to a sealed or sealable reaction vessel and have chemical compatibility for a wide range of solvent properties. The liquid handling device specifically manufactured for organic syntheses are the most desirable for application to crystallization due to the chemical compatibility issues. Robbins Scientific manufactures the Flexchem reaction block which consists of a Teflon reaction block with removable gasketed top and bottom plates. This reaction block is in the standard footprint of a 96-well microtiter plate and provides for individually sealed reaction chambers for each well. The gasketing material is typically Viton, neoprene/Viton, or Teflon coated Viton, and acts as a septum to seal each well. As a result, the pipetting tips of the liquid handling system need to have septum-piercing capability. The Flexchem reaction vessel is designed to be reusable in that the reaction block can be cleaned and reused with new gasket material.

An array can be prepared, processed, and screened as follows. The first step comprises selecting the component sources, preferably, at one or more concentrations. Preferably, at least one component source can deliver a compound-of-interest and one can deliver a solvent. Next, adding the compound-of-interest and components to a plurality of sample sites, such as sample wells or sample tubes on a sample plate to give an array of unprocessed samples. The array can then be processed according to the purpose and objective of the experiment, and one of skill in the art will readily ascertain the appropriate processing conditions. Preferably, the automated distribution mechanism as described above is used to distribute or add components.

Once an array is prepared, solid formation can be induced by introducing a nucleation or precipitation event. In general, this involves subjecting a supersaturated solution to some form of energy, such as ultrasound or mechanical stimulation or by inducing supersaturation by adding additional components.

The array can be processed according to the design and objective of the experiment. One of skill in the art will readily ascertain the appropriate processing conditions. Processing includes mixing; agitating; heating; cooling; adjusting the pressure; adding additional components, such as crystallization aids, nucleation promoters, nucleation inhibitors, acids, or bases, etc.; stirring; milling; filtering; centrifuging, emulsifying, subjecting one or more of the samples to mechanical stimulation; ultrasound; or laser energy; or subjection the samples to temperature gradient or simply allowing the samples to stand for a period of time at a specified temperature. A few of the more important processing parameters are elaborated below.

In some array experiments, processing will comprise dissolving either the compound-of-interest or one or more components. Solubility is commonly controlled by the composition (identity of components and/or the compound-of-interest) or by the temperature. The latter is most common in industrial crystallizers where a solution of a substance is cooled from a state in which it is freely soluble to one where the solubility is exceeded. For example, the array can be processed by heating to a temperature (T1), referably to a temperature at which the all the solids are completely in solution. The samples are then cooled, to a lower temperature (T2). The presence of solids can then determined. Implementation of this approach in arrays can be done on an individual sample site basis or for the entire array (i.e., all the samples in parallel). For example, each sample site could be warmed by local heating to a point at which the components and the compound-of-interest are dissolved. This step is followed by cooling through local thermal conduction or convection. A temperature sensor in each sample site can be used to record the temperature when the first crystal or precipitate is detected. In one embodiment, all the sample sites are processed individually with respect to temperature and small heaters, cooling coils, and temperature sensors for each sample site are provided and controlled. This approach is useful if each sample site has the same composition and the experiment is designed to sample a large number of temperature profiles to find those profiles that produce desired solid-forms. In another embodiment, the composition of each sample site is controlled and the entire array is heated and cooled as a unit. The advantage of the latter approach is that much simpler heating, cooling, and controlling systems can be utilized. Alternatively, thermal profiles are investigated by simultaneous experiments on identical array stages. Thus, a high-throughput matrix of experiments in both composition and thermal profiles can be obtained by parallel operation.

Typically, several distinct temperatures are tested during crystal nucleation and growth phases. Temperature can be controlled in either a static or dynamic manner. Static temperature means that a set incubation temperature is used throughout the experiment. Alternatively, a temperature gradient can be used. For example, the temperature can be lowered at a certain rate throughout the experiment. Furthermore, temperature can be controlled in a way as to have both static and dynamic components. For example, a constant temperature (e.g., 60° C.) is maintained during the mixing of crystallization reagents. After mixing of reagents is complete, controlled temperature decline is initiated (e.g., 60° C. to about 25° C. over 35 minutes).

Stand-alone devices employing Peltier-effect cooling and joule-heating are commercially available for use with microtiter plate footprints. A standard thermocycler used for PCR, such as those manufactured by MJ Research or PE Biosystems, can also be used to accomplish the temperature control. The use of these devices, however, necessitates the use of conical vials of conical bottom micro-well plates. If greater throughput or increased user autonomy is required, then full-scale systems such as the advanced Chemtech Benchmark Omega 96TM or Venture 596 TM would be the platforms of choice. Both of these platforms utilize 96-well reaction blocks made from Teflon™. These reaction blocks can be rapidly and precisely controlled from −70 to 150° C. with complete isolation between individual wells. Also, both systems operate under inert atmospheres of nitrogen or argon and utilize all chemically inert liquid handling elements. The Omega 496 system has simultaneous independent dual coaxial probes for liquid handling, while the Venture 596 system has 2 independent 8-channel probe heads with independent z-control. Moreover, the Venture 596 system can process up to 10,000 reactions simultaneously. Both systems offer complete autonomy of operation.

Array samples can be incubated for various lengths of time (e.g., 5 minutes, 60 minutes, 48 hours, etc.). Since phase changes can be time dependent, it can be advantageous to monitors arrays experiments as a function of time. Im many cases, time control is very important, for example, the first solid-form to crystallize may not be the most stable, but rather a metastable form which can then convert to a form stable over a period of time. This process is called “ageing”. Ageing also can be associated with changes in crystal size and/or habit. This type of ageing phenomena is called Ostwald ripening.

The pH of the sample medium can determine the physical state and properties of the solid phase that is generated. The pH can be controlled by the addition of inorganic and organic acids and bases. The pH of samples can be monitored with standard pH meters modified according to the volume of the sample.

The following discussion describes a number of preferred embodiments of the present invention, no part of which should be construed as limiting the present invention in any way.

In one preferred embodiment of the present invention, the system is used in conjunction with one or more high-throughput automated experimentation apparatus, such as Transform Pharmaceutical's FAST™ formulation system or CRYSTALMAX™ crystal discovery system. The FAST and CRYSTALMAX systems are described in U.S. patent application Ser. Nos. 09/628,667 and 09/756,092, respectively, (the FAST™ and CRYSTALMAX™ applications) which are incorporated herein by reference. Words used herein are intended to be consistent with the FAST™ and CRYSTALMAX™ applications. In this embodiment, the system is used to plan and assess experiments performed with the CRYSTALMAX™ and FAST™ systems.

This embodiment includes a process informatics subsystem for controlling and acquiring data from the CRYSTALMAX and FAST systems, and a computational informatics subsystem for performing data mining, simulation, molecular modeling, high-dimensional multivariate visualizations of data, data clustering, categorizations, and other data processing. These subsystems operate on a shared database system used to store experimental results and analyses, as well as data derived from sources other than the process informatics subsystem, such as external databases and literature.

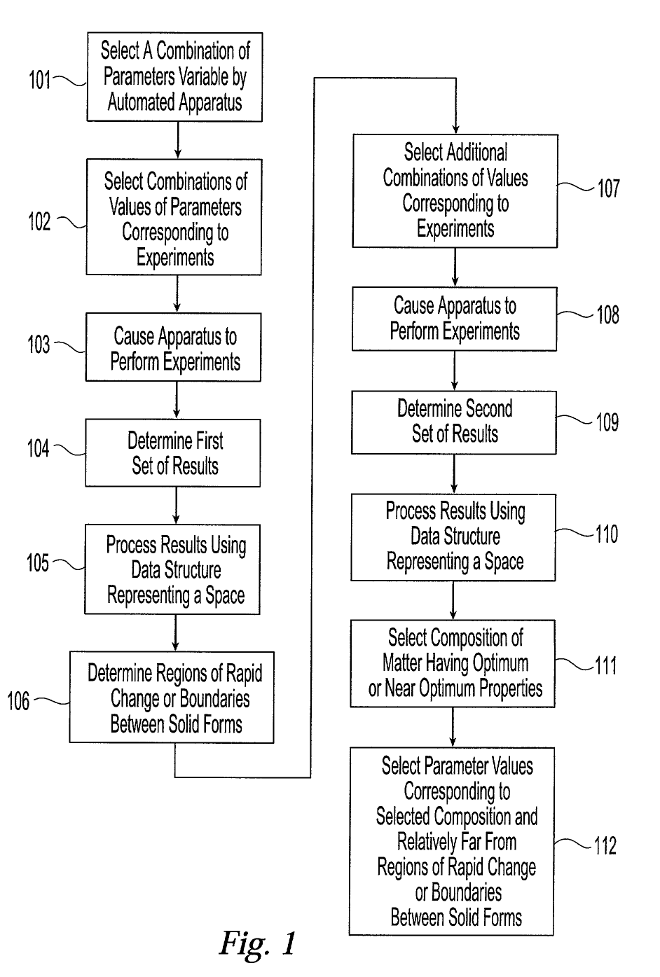

As schematically illustrated in FIG. 1, using the computational informatics subsystem, a combination of experimental parameters which may be varied by an automated experimentation apparatus such as FAST or CRYSTALMAX is selected 101. A first plurality of distinct combinations of values of the experimental parameters is then determined, each combination corresponding to a distinct experiment 102. Using the process informatics subsystem, the automated experimentation apparatus is caused to conduct a first set of experiments, each experiment of the first set corresponding to a distinct combination of the first plurality of distinct combinations 103. The process informatics subsystem is also used to determine a first collection of experimental results of the first set of experiments, the first collection comprising a plurality of individual result sets, where each individual result set corresponds to a distinct experiment 104.

The first collection of experimental results is processed through the computational informatics subsystem to determine a second plurality of distinct combinations of values of the experimental parameters, each combination of the second plurality corresponding to a distinct experiment.

Preferably, data representing the first collection of experimental results is processed as a collection of points in a space, such as a topological space, a metric space, or a vector space comprising dimensions corresponding to the dimensions of the experimental parameters 105. Through such analysis, regions of the space are determined in which significant changes in result sets occur in connection with relatively small changes in the experimental parameters. For example, boundaries between solid forms, or regions in which desired properties of formulations change rapidly with experimental parameters are preferably identified 106. Based on this identification, the second plurality of distinct combinations of values of the experimental parameters is preferably selected 107 to more fully define such boundaries or regions, and to include combinations of parameters as far as possible from such boundaries or regions.

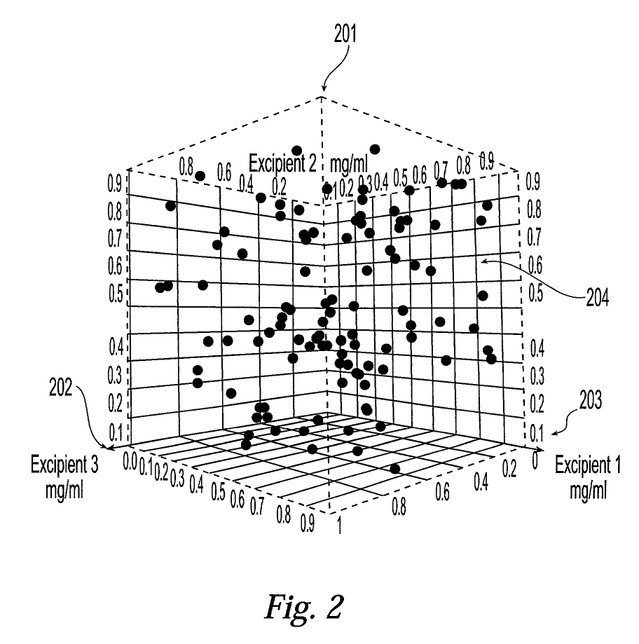

For example, the first collection of experimental results is preferably processed by the computational informatics subsystem to display a high-dimensional visualization in which the experimental results are represented as points of varying size, color, shape or other indicia in a multidimensional space representing the space or a projection thereof such as that shown in FIG. 2. By viewing such a visualization, an operator of the computational informatics subsystem may visually identify boundaries or regions of rapid change.

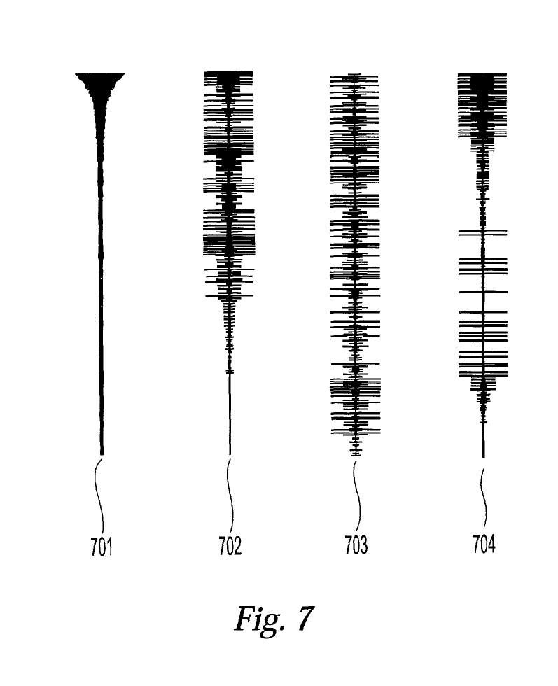

Alternatively, or in addition, other forms of multivariate visualization may be used, such as that depicted in FIG. 7. FIG. 7 depicts a multivariate visualization of one thousand experimental formulations, each with three excipients and one measured property of the formulation. Four axes 701, 702, 703 and 704 are depicted. Distinct formulations appear along the length of the axes, with each formulation appearing at the same place along all four axes. The width of each line on each axis is proportional to the normalized magnitude of the value represented by the axis for the corresponding experiment. For example, concentrations of excipients may be shown by the widths along axes 702, 703, and 704 and solubility may be shown by the widths along 701.

Alternatively, or in addition, combinations of values of the second plurality may be selected along a line or curve fitted to the data using any regression method. Other examples of selection include random or uniform selection within a range of values for results exhibiting desired properties, or selection within a range determined by use of one or more classification algorithms, such as a range classified as likely to correspond to a single solid form, or a range classified as likely to include a boundary between sets of experimental conditions within which two distinct solid forms are produced. Selection of additional values may also include a change of experimental parameters such as selection of different reagents or excipients likely to interact with observed species or solid forms. These and other preferred methods comprise other aspects of the invention, and are discussed in greater detail below.

Using the process informatics subsystem, the automated experimentation apparatus is activated to conduct a second set of experiments, each experiment of the second set corresponding to a distinct combination of values of the second plurality 108. The process informatics subsystem is also used to determine a second collection of experimental results of the second set of experiments, the second collection comprising a plurality of individual results, each individual result corresponding to a distinct experiment 109.

The computational informatics subsystem is then used to select a multicomponent chemical composition of matter based on the first collection of experimental results and the second collection of experimental results. Alternatively, additional iterations of experimentation may be performed prior to selecting the multicomponent chemical composition.

As with the prior collection of experimental results, data representing the second or subsequent collection of experimental results is preferably processed as a collection of points in a space such as topological space, metric space, or vector space comprising dimensions corresponding to the dimensions of the experimental parameters 110. Based on this processing, a set of experimental parameter values and a resulting multicomponent chemical composition of matter is preferably selected having optimum or near-optimum properties that do not change significantly within a region of the space corresponding to an expected range of conditions of manufacture, storage, and administration or use 111.

Planning and Assessing a Search for an Optimized Formulation

In one example preferred embodiment, an experimental search for a formulation having an optimized solubility is performed. This example is schematically illustrated in FIGS. 2-4. First, a combination of experimental parameters which may be varied by an automated experimentation apparatus is selected. In this example embodiment, the selected experimental parameters are concentrations of three selected excipients, schematically illustrated as a three-dimensional metric space in FIG. 2 comprising axes 201, 202, 203 of plot 204. A first plurality of distinct combinations of values of the experimental parameters is then determined, each combination corresponding to a distinct experiment. The combinations of values correspond to the coordinates of each of the data points 204 shown in FIG. 2.

Using the process informatics subsystem, the automated experimentation apparatus is caused to conduct a first set of experiments, each experiment of the first set corresponding to a distinct combination of the first plurality. In this example embodiment, each experiment comprises a sample formulation. Each sample formulation comprises one or more target active agents at fixed concentrations and a combination of excipients having concentrations corresponding to one of the data points 204 of FIG. 2. The process informatics subsystem is also used to determine a first collection of experimental results of the first set of experiments, the first collection comprising a plurality of individual result sets, where each individual result set corresponds to a distinct experiment. Each individual result set in this example embodiment comprises a measurement of the amount of an active component dissolved using standard technology such as mass spectroscopy, HPLC, UV spectroscopy, fluorescence spectroscopy, gas chromatography, optical density or colorimetry.

Using the process informatics subsystem, the measured experimental results are stored in a shared database, and thereby made available to the computational informatics subsystem. The computational informatics subsystem may then be used to visualize the experimental data in a high-dimensional multivariate display. In the display illustrated in FIG. 2, the size of plotted data points are used to depict the measured solubility of the active portion of the formulations corresponding to the data points 204, wherein larger sizes indicate greater solubility.

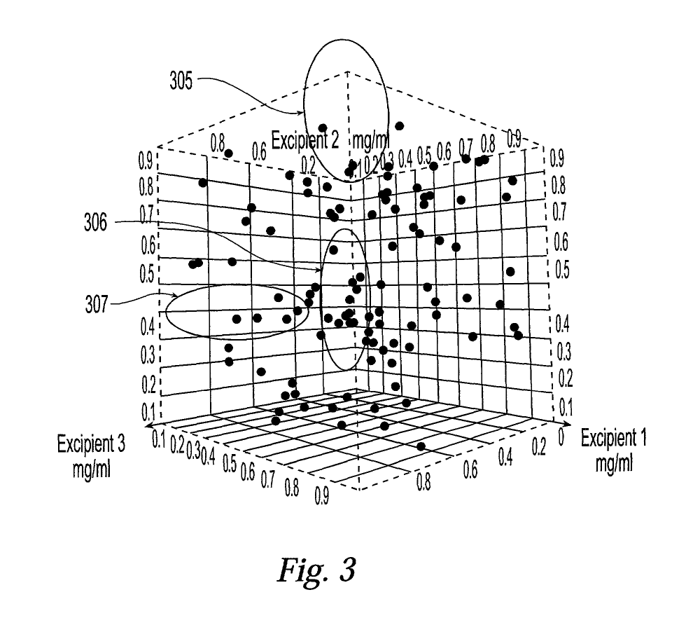

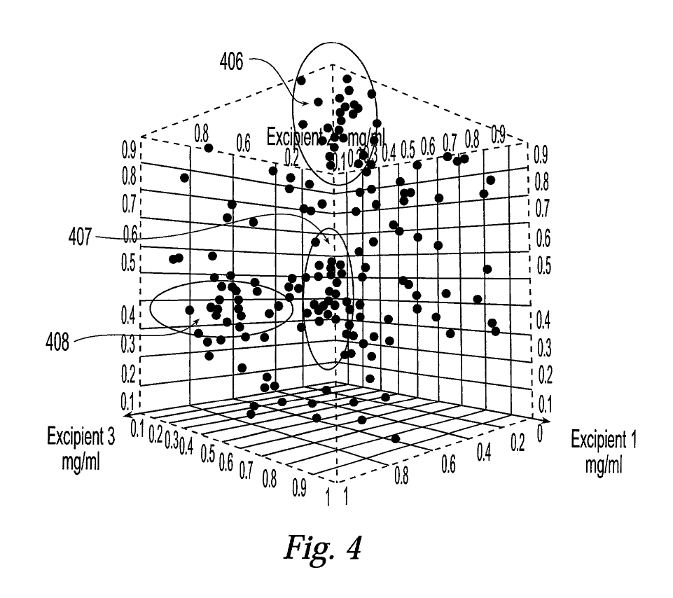

Using the computational informatics subsystem, a second plurality of distinct combinations of values of the experimental parameters is determined, based on the measured experimental results. For example, as shown in FIG. 3, certain experimental results or groups of experimental results 305, 306, 307 are identified as exhibiting measured results of interest. As shown in FIG. 4, additional data points 406, 407, 408 corresponding to distinct experiments may be selected to more accurately characterize the formulation near the results of interest. In this example embodiment, a portion of the experimental results 305 of interest are solubility maxima or near-maxima in the sample. Another portion of the results of interest are groups of results 306 for which the rate of solubility change with respect to one or more experimental parameters is high relative to other groups of the sample. In this case, more experiments 406, 407 in this region will more accurately characterize the relationship between the experimental parameters and the change in solubility in the region. A third set of results of interest in this example are results 307 for which the rate of change of solubility with respect to one or more experimental parameters is low relative to other groups of the sample. In this situation, it is desirable to verify that the rate of change is low throughout the region by performing experiments 408 at a greater resolution to ensure that no changes in solubility have been missed by the resolution of the first set of experiments. Greater resolution is achieved by spacing the experiments 408 more densely in the region.

Using the process informatics subsystem, the automated experimentation apparatus is activated to conduct a second set of experiments, each experiment of the second set corresponding to a selected additional data point. In this example embodiment, each experiment comprises a sample formulation of the same one or more active agents and excipients as the first set of experiments. Alternatively, the concentration or identity of the one or more target active agents, or the identities of one or more excipients could be changed for the second set of experiments. The process informatics subsystem is also used to determine a second collection of experimental results of the second set of experiments. In this example embodiment, the same measurement of solubility used for the first set of experiments is performed for the second set. Alternatively, a different measurement could be used for the second set.

Using the process informatics subsystem, measured experimental results are stored in the shared database, and thereby made available to the computational informatics subsystem. The computational informatics subsystem may then be used to visualize the experimental data in a high-dimensional display. In the display illustrated in FIG. 4, the size of plotted data points are used to depict the measured solubility of the active portion of the formulations corresponding to the data points of the second set of experiments 406.

Additional iterations of selecting additional data points and automated experimentation may be performed. Based on the collection of results, an optimum formulation is selected. In this solubility example, an optimum formulation is one having a high relative solubility, but comprising a combination of concentrations of excipients away from areas in which solubility changes relatively rapidly with concentration of one or more excipients. By avoiding a formulation in areas of rapid change, changes in the properties of the formulation due to expected variations of conditions of manufacture, storage, and administration or use are minimized.

Planning and Assessing a Massively Parallel Search for New Solid Forms

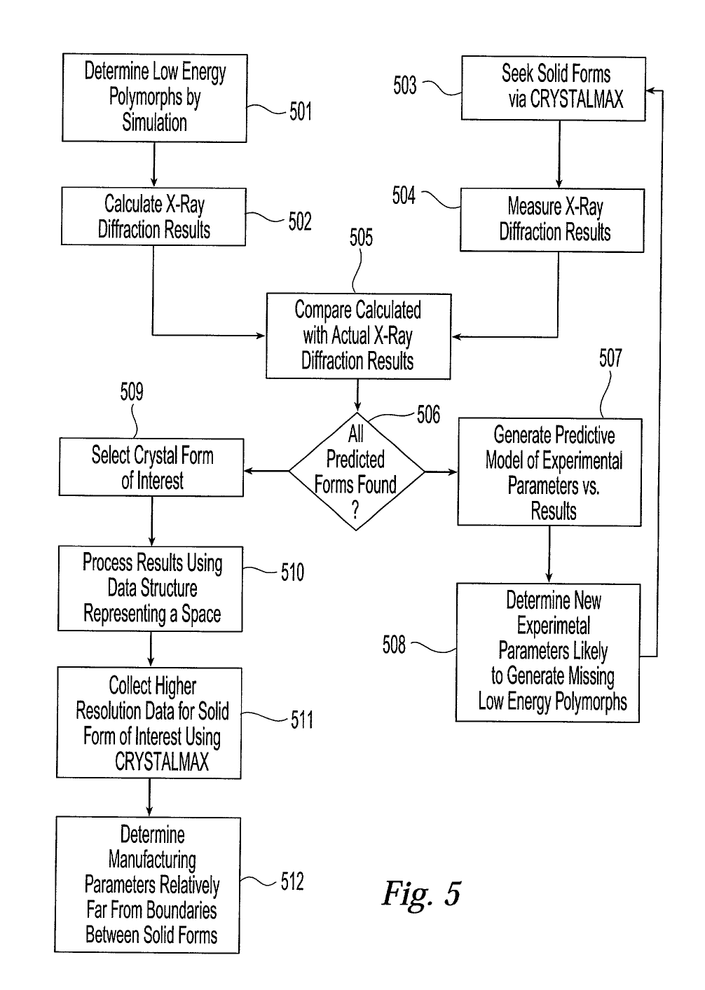

A preferred method to assess the first collection of experimental results in a search for novel or known solid forms is schematically illustrated in FIG. 5. The method comprises the steps of: determining low-energy crystal polymorphs via simulation 501; characterizing the low-energy crystal polymorphs according to expected experimental results by standard techniques such as by calculated X-ray powder or single-crystal diffraction results 502; conducting a first collection of crystallization experiments 503; measuring a collection of actual experimental results such as actual X-ray powder diffraction for the crystals produced by the first collection of crystallization experiments 504; comparing the expected experimental results with the actual experimental results 505; determining if any lowest-energy structures were not included in the solid forms produced by a first collection of experiments 506.

Preferably, low-energy polymorphs are determined by using multivariate optimization such as hydrogen-bond-biased simulated annealing to locate a plurality of lowest-energy structures with the model. One preferred energy function is crystal lattice energy, also referred to as the crystal binding or cohesive energy. Lattice energy is determined by summing all the pairwise atom-atom interactions between a central molecule and all the surrounding molecules. The calculation of lattice energy is discussed in Myerson, Molecular Modeling Applications in Crystallization, pp. 117-125, Cambridge University Press (1999), which is incorporated herein in its entirety by reference. The lattice energy is a useful parameter because its calculated value can be compared with the experimental enthalpy of sublimation. This allows one to verify the description of the intermolecular interactions by the force field in question.

An advantage of the calculated value of the crystal lattice energy is that it can be separated into specific interactions along certain directions and into the constituent atom-atom pairwise contributions. This provides the link between molecular and crystal structures. The calculation of lattice energies thus provides a profile of the important intermolecular interactions that correspond to particular classes of compounds. It also provides an understanding of the nature of the intermolecular interactions that lead to a particular crystal packing arrangement.

Preferably, in performing atom-atom interactions, the potentials used include those that incorporate attractive or repulsive components, coulombic interaction, or hydrogen-bonding interaction. An example of these potentials include the Lennard-Jones potential, V_{vdw}=−A/r⁶+B/r¹², where A and B are the atom-atom parameters and r is the interatomic distance. The parameters A and B can be obtained by fitting the chosen potential to observable properties such as crystal structure, heats of sublimation, and hardness measurements. In accordance with the present invention, the results of a first principles calculation can also be used in the curve fitting step as an alternative to using actual experimental data to determine the parameters A and B. The coulombic interaction may be calculated using the equation V_{coul}=qᵢqⱼ/(Dr) where qᵢ and qⱼ are the charges on atoms i and j, D is the dielectric constant, and r is the interatomic distance. The hydrogen bonding potential may be calculated using a modified form of van der Waals potential such as a V_{vdw}=−A/r¹⁰+B/r¹² potential instead of V_{vdw}=−A/r⁶+B/r¹² for the commonly used van der Waals potential function.

An example preferred multivariate optimization method used to search for a low energy crystal structure is the hydrogen-bond-biased simulated annealing monte carlo (SAMC) method described by Chin and co-workers in J. Am Chem. Soc. 1999, 121, 2115-2122, the entirety of which is incorporated herein by reference. As described therein, one first builds and parameterizes a molecule using a molecular modeling program such as QUANTA, available from Molecular Simulations Inc., and then minimizes its energy using a program such as CHARMm, also available from Molecular Simulations Inc. (an academic version of the program, referred to as CHARMM, is also available from Harvard University). The molecular frame of reference is preferably positioned at the molecule's center of mass. Using preset limits of the unit cell and molecular rotation, a trial crystal structure with a given space group is built using a program such as CHARMM. Preferably, the limits used are: (a) a “loose” window for the lengths of the axes of the unit cell (for example, 30% greater than the largest molecular dimension as an upper limit and 3% less than the smallest dimension of the molecule as the lower limit); and (b) a range of angles corresponding to the allowable degree of molecular rotation.

Preferably, the above limits are chosen to ensure that any van der Waals interaction or contact present in the initially found crystal structure is not energetically unfavorable. In a preferred SAMC method and minimization procedure, the number of energetically favorable van der Waals interactions between the molecule and its crystalline environment increases with the lowering of the simulated annealing temperature.

To calculate the crystal energy (CE) of the trial crystal structure, CE can be expressed in terms of a Lennard-Jones type potential function and a coulombic interaction potential such as the one shown below.

CE=Σᵢⱼ[Aᵢⱼ/rᵢⱼ¹²−Bᵢⱼ/rᵢⱼ⁶+qᵢqⱼ/4π∈ₒrᵢⱼ]

Thus, summing the contributions of the pairwise interactions between the i^{th} atom on the initial molecule and the j^{th} atom of the surrounding molecules in the crystal allows calculation of CE. In the CE expression given above, rᵢⱼ is the distance between atoms i and j, Aᵢⱼ=(AᵢAⱼ)^{½} (for example) and Bᵢⱼ are the van der Waals parameters corresponding to atoms i and j, qᵢ represents the partial atomic charge of atom i, and ∈ₒ is the permittivity of free space (8.854×10⁻¹² C²/Nm²).

In a preferred embodiment, unit cell dimensions are used as variables to be searched in the presence of the crystalline environment, and structures are chosen based on whether or not their hydrogen-bonding energies exceed a given value.

The SAMC method may be summarized as follows:

(1) building, parameterizing, and minimizing the energy of a molecule that will be used for the crystal construction.

(2) creating a reference crystal structure based on the molecule created in step (1) by randomly varying the unit cell parameters appropriate for the given crystal space group and the preselected molecular rotational constraint.

(3) calculating the crystal energy of the reference crystal and setting the value obtained as CE₀.

(4) generating another crystal as in step (2) based on the given molecular constraints.

(5) minimizing the crystal energy using a gradient-descent method until the energy gradient falls below a certain limit.

(6) calculating the hydrogen-bonding energy of the energy-minimized crystal.

(7) rejecting the energy-minimized crystal if its hydrogen-bonding energy is greater than or equal to zero.

(8) denoting the crystal energy of the energy-minimized crystal as CE₁ if its hydrogen-bonding energy is less than zero.

(9) comparing CE₁ with that of the previous crystal (CEO in the first iteration).

(10) setting CE₀=CE₁ if CE₁<CE₀.

(11) if CE₁>CE₀, calculating the Boltzmann weighting factor at a given temperature T, where the weighting factor is expressed by W=exp[−(CE₁−CE₀/k_{b}T)] where k_{b} is the Boltzmann constant and T is assigned an initial value (4,300° K, for example).

(12) generating a random number R between 0 and 1.

(13) comparing W in obtained in step (11) with the generated random number R in (12).

(14) rejecting the crystal structure corresponding to CE₁ if R≧W.

(15) setting CE₀=CE₁ if R<W.

(16) repeating steps 4-14 until a certain number of crystal structures have been obtained for a given temperature T.

(17) lowering the temperature by a certain value (500° K, for example), and repeating the entire procedure beginning with the last structure collected from the previous temperature.

(18) repeating steps 2-17 until the temperature has dropped below a given value.

(19) ranking all the structures collected at various temperatures from the lowest energy to the highest.

(20) selecting a plurality of the lowest energy structures from the ranking.

After selecting a plurality of the lowest energy structures from the ranking, the selected structures are characterized, according to expected experimental results, for solid forms corresponding to the structures. Preferably, the lowest energy structures are characterized by calculating X-ray powder diffraction results for each structure. Software for calculating X-ray powder diffraction results from a known structure, known as Cerius2, is available from Molecular Simulations Inc.

After characterizing the lowest energy structures according to expected experimental results, the process informatics subsystem preferably compares the expected experimental results with a set of actual experimental results from a first set of experiments. Based on the comparison, the process informatics subsystem assesses which, if any, of the lowest energy structures was produced by each experiment.

Preferably, the process informatics subsystem compares the expected experimental results with the actual experimental results of the first set of experiments by comparing calculated X-ray powder diffraction results for the lowest energy structures with experimentally measured X-ray powder diffraction results for the first set of experiments. The comparison is preferably performed by calculating a similarity measure of the expected experimental results and the actual experimentally measured results. Preferably, the similarity measure is calculated as

SI=d·F·d

where

d=s₁−s₂

is an n-vector that describes the difference between normalized sets of points in the calculated X-ray powder diffraction pattern s, and the measured X-ray powder diffraction s₂. And

F={F_{i,j}}

Where F is the n×n “fold matrix” described in Karfunkel, H. R.; Rohde, B.; Leusen, F. J. J.; Gdanitz, R. J.; Rihs, G. J. Comput. Chem. 1993, 14, 1125-1135, which is hereby incorporated in its entirety by reference. Specifically,

F_{i,j}=1/(1+α(i−j)^{β})

where

the values of α and β are those empirically calibrated by Karfunkel: α=1.0×10⁻⁸ and β=4.

The similarity measure SI compares each point in one set with the set of nearby points in the other set, giving decreasing weight to points further away. Two identical sets would have an SI of zero. Larger values of SI imply greater dissimilarity between the calculated and measured spectra. This similarity measure was used by Chin and co-workers in the reference cited above and incorporated herein by reference.

Other forms of similarity measure may be used, however, it is preferable to use a measure that accounts for similarity over a neighborhood. Nevertheless, simpler methods such as the mean-square-difference between the two patterns may be used.

Based on the similarity measure, each experiment is classified as to which predicted lowest energy form was produced. This may be accomplished by classifying each experiment as the predicted low-energy structure having a calculated X-ray powder diffraction pattern most similar to the measured X-ray powder diffraction pattern according to the similarity measure applied. Using the preferred similarity measure, each experiment is classified as the predicted low-energy structure for which SI measure is the least. Preferably, a threshold is also applied, so that measured patterns for which the least SI is above the threshold are classified as “unknown.”

One preferred way of planning additional experiments to find missing expected solid forms is schematically illustrated in FIG. 5: generating a predictive model, such as a regression model, of the experimental parameters and results from the first set of experiments 507, and interpolating or extrapolating those results to determine sets of experimental parameters likely to produce predicted low-energy structures not produced in the first set of experiments 508.

One preferred method for generating a predictive model from the first set of experimental results is to apply Multivariate Adaptive Regression Splines (MARS) to the classified experimental results from the first set of experiments. MARS is described in J. H. Friedman, Multivariate Adaptive Regression Splines, SLAC PUB-4960 Rev, Tech Report 102 Rev (Stanford Linear Accelerator Center, 1990) at http://www.slac.stanford.edu/pubs/slacpubs/4750/slac-pub-4960.pdf which is incorporated herein by reference in its entirety. A computerized implementation of MARS is commercially available from Salford Systems of San Diego, Calif. (www.salfordsystems.com). Other regression methods such as linear regression, stepwise linear regression, additive models (AM), projection pursuit regression (PPR), recursive partitioning regression (RPR), alternating conditional expectations (ACE), additivity and variance stabilization (AVAS), locally weighted regression (LOESS), and neural networks may also be used.

After generating a predictive model, the model is used to determine a second set of distinct combinations of experimental parameters that, according to the model, should produce predicted solid forms that were not produced in the first set of experiments. This may be accomplished by setting the response variable to a value corresponding to a missing predicted solid form and solving the predictive model for one or more sets of values of experimental parameters giving that result. For preferred predictive models, the solution may be found using algebraic or numerical methods readily apparent to those of ordinary skill in the art of using such predictive models.

Using the process informatics subsystem, the automated experimentation apparatus is activated to conduct a second set of experiments, each experiment of the second set corresponding to a distinct combination of experimental parameters determined using the predictive model. The second set of experimental results are preferably again compared against predicted experimental results as described above to classify the results according to predicted solid forms and to determine if all predicted low-energy structures have been produced.

Based on the collection of results, an optimum or near-optimum solid form is selected 509. Preferably, data representing the collection of experimental results is processed as a collection of points in a space, such as a topological space, metric space, or vector space comprising dimensions corresponding to the dimensions of the experimental parameters 510. Through such analysis, regions of the space in which the selected solid form is produced, and the boundaries between such regions and regions in which other forms or no solid forms are produced may be determined. Additional sets of experiments may be performed to define such regions with greater resolution 511. Preferably set of experimental parameters is thereby determined as far as possible from such boundaries 512. Such a set of parameters is advantageous for manufacture because small variations in manufacturing conditions are less likely to produce a solid form other than the selected form.

Preferred Embodiments of Process Informatics and Computational Informatics Subsystems

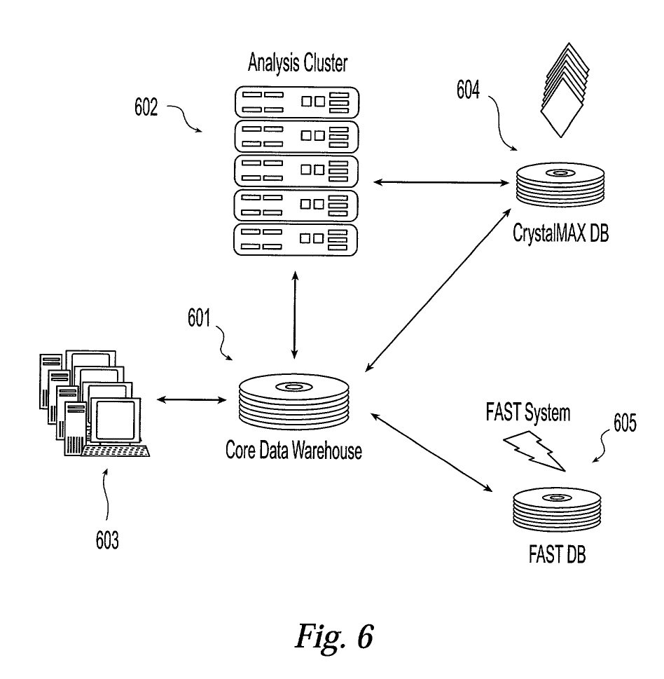

The architecture of a preferred example embodiment is schematically illustrated in FIG. 6. The computational informatics subsystem comprises a core data warehouse 601 and analysis cluster 602. The core data warehouse 601 comprises an Oracle 8i object-oriented relational database management system with partitioning option running under Linux on a Penguin Computing Systems 8500 computer with eight Intel Pentium III 550 megahertz Xeon CPUs and 2 gigabytes of RAM and a one terabyte RAID 5 disk array. The analysis cluster 602 comprises a Penguin Computing Systems Blackfoot dual Intel Pentium III 800 megahertz CPUs with 2 Gigabytes of RAM and 36 gigabytes of disk space running Linux with the MOSIX kernel modification.

The process informatics subsystem comprises a CRYSTALMAX informatics system 604 and a FAST informatics system 605. The CRYSTALMAX informatics system 604 comprises an Oracle 8i object-oriented relational database management system running under Linux on a Penguin Computing Systems 4400 with 4 Intel Pentium Xeon CPUs, 2 gigabytes of RAM and a 500 gigabyte RAID 5 disk array. The FAST informatics system 605 has the same configuration.

Windows systems 603 preferably comprise a variety of personal workstation hardware ranging from typical desktop PCs to high-performance workstations with visualization hardware.

The core data warehouse 601 and analysis cluster 602 are preferably interconnected with gigabit Ethernet. The CRYSTALMAX 604 and FAST 605 informatics systems are also preferably interconnected with the computational informatics subsystem by gigabit Ethernet. Windows systems 603 are typically connected to the computational informatics subsystem by a variety of heterogenous networks, including the Internet.

What is claimed is:

1. A method for determining one or more multicomponent chemical compositions, comprising the steps of:
selecting a combination of experimental parameters that may be varied by a high-throughput automated experimentation apparatus;
determining a first plurality of distinct combinations of values of the experimental parameters, each combination corresponding to a distinct experiment;
causing the automated experimentation apparatus to conduct a first set of experiments for each of at least a portion of the first plurality of distinct combinations of values of the experimental parameters;
determining a first collection of experimental results of the first set of experiments, the first collection comprising a plurality of individual result sets, each individual result set corresponding to a distinct experiment;
based on the first collection of experimental results, determining a second plurality of distinct combinations of values of the experimental parameters, each combination corresponding to a distinct experiment;
causing the automated experimentation apparatus to conduct a second set of experiments for each of at least a portion of the second plurality of distinct combinations of values of the experimental parameters;
determining a second collection of experimental results of the second set of experiments, the second collection comprising a plurality of individual result sets, each individual result set corresponding to a distinct experiment;
selecting one or more multicomponent chemical compositions of matter based on the first collection of experimental results and the second collection of experimental results.

2. A method for determining one or more solid forms of a compound, comprising the steps of:
selecting a combination of experimental parameters that may be varied by a high-throughput automated experimentation apparatus;
determining a first plurality of distinct combinations of values of the experimental parameters, each combination corresponding to a distinct experiment;
causing the automated experimentation apparatus to conduct a first set of experiments for each of at least a portion of the first plurality of distinct combinations of values of the experimental parameters;
determining a first collection of experimental results of the first set of experiments, the first collection comprising a plurality of individual result sets, each individual result set corresponding to a distinct experiment;
based on the first collection of experimental results, determining a second plurality of distinct combinations of values of the experimental parameters, each combination corresponding to a distinct experiment;
causing the automated experimentation apparatus to conduct a second set of experiments for each of at least a portion of the second plurality of distinct combinations of values of the experimental parameters;
determining a second collection of experimental results of the second set of experiments, the second collection comprising a plurality of individual result sets, each individual result set corresponding to a distinct experiment;
selecting one or more solid forms based on the first collection of experimental results and the second collection of experimental results.

3. The method of claim 1, wherein the second plurality of distinct combinations of values of experimental parameters is determined based on a comparison of differences of results corresponding to experiments from the first set of experiments with differences of experimental parameters corresponding to the same experiments.

4. The method of claim 3 wherein the comparison comprises a measure of change of experimental results relative to at least one experimental parameter.

5. The method of claim 4 wherein at least a portion of the second plurality of values of the experimental parameters are selected from a range of experimental parameters including values corresponding to a first subset of experiments from the first set of experiments for which the measure of change of experimental results relative to at least one experimental parameter is higher than the measure of change for a second subset of experiments from the first set of experiments.

6. The method of claim 4 wherein at least a portion of the second plurality of values of the experimental parameters are selected from a range of experimental parameters including values corresponding to a first subset of experiments from the first set of experiments for which the measure of change of experimental results relative to at least one experimental parameter is lower than the measure of change for a second subset of experiments from the first set of experiments.

7. The method of claim 1 or 2 wherein at least a portion of the second plurality of distinct combinations of values of experimental parameters is determined based on a comparison of individual results corresponding to experiments from the first set of experiments with one or more target values.

8. The method of claim 1 or 2 wherein at least a portion of the second plurality of distinct combinations of values of experimental parameters is determined based on at least one database query derived from one or more experimental results from the first collection of experimental results.

9. The method of claim 1 or 2, wherein at least a portion of the second plurality of distinct combinations of values of experimental parameters is determined based on the output of at least one simulation that used as input at least one experimental result from the first collection of experimental results.

10. The method of claim 8 wherein the query comprises one or more molecular descriptors characteristic of at least one experiment selected from the first set of experiments.

11. A method of estimating one or more properties of a multicomponent chemical composition comprising the steps of:
receiving signals representing an experimental result set for each of a plurality of experiments conducted by a high-throughput automated experimentation apparatus;
for each of at least a portion of the experiments, generating a predictive model based on signals characterizing each experimental result set according to the property to be estimated and signals characterizing the experiment with respect to a set of molecular descriptors;
estimating the property for a multicomponent chemical composition by providing signals characterizing the multicomponent chemical composition with respect to the molecular descriptors as input to the predictive model.

12. A method of estimating a property of a solid form of a compound, comprising the steps of:
receiving signals representing experimental result sets for a plurality of experiments conducted by a high-throughput automated experimentation apparatus;
generating a predictive model based on signals characterizing at least a portion of the experimental result sets according to the property to be estimated and signals characterizing the experiments with respect to a set of molecular descriptors;
estimating the property for a solid form of a compound by providing signals characterizing the solid form of the compound with respect to the molecular descriptors as input to the predictive model.

13. A method of estimating a property of a multicomponent chemical composition comprising the steps of:
receiving signals representing a simulation result set for each of a plurality of simulations of a compound;
for at least a portion of the simulations, generating a predictive model based on signals characterizing each simulation result set according to the property to be estimated and signals characterizing the simulation results sets with respect to a set of molecular descriptors;
receiving signals representing experimental result sets for a plurality of experiments conducted by a high-throughput automated experimentation apparatus;
estimating the property for the multicomponent chemical composition by providing signals characterizing the multicomponent chemical composition with respect to the set of molecular descriptors as input to the predictive model.

14. A method of estimating a property of a solid form of a compound comprising the steps of:
receiving signals representing simulation result sets for a plurality of simulations of a compound;
for at least a portion of the simulations, generating a predictive model based on signals characterizing the simulation result sets according to the property to be estimated and signals characterizing the simulation result sets with respect to a set of molecular descriptors;
receiving signals representing experimental result sets for a plurality of experiments conducted by a high-throughput automated experimentation apparatus;
estimating the property for the solid form of the compound by providing signals characterizing the solid form of the compound with respect to the set of molecular descriptors as input to the predictive model.

15. A method of determining a multicomponent chemical composition of matter, comprising the method of claim 11, 12, 13 or 14, and further comprising the steps of:
based on the estimation, determining a plurality of distinct combinations of values of the experimental parameters, each combination corresponding to a distinct experiment;
causing the automated experimentation apparatus to conduct a second plurality of experiments corresponding to the plurality of distinct combinations of values of the experimental parameters;
receiving signals representing an experimental result set for each of the second plurality of experiments;
selecting a multicomponent chemical composition of matter based on the experimental result sets.

16. The method of claim 2 wherein the second plurality of distinct combinations of values of experimental parameters is determined based on hydrogen-bond-biased simulated annealing monte carlo screening.

17. A multicomponent chemical composition of matter determined using the method of claim 1, 2, 3, 4, 5, 6, 7, 8, 10, 11, 12, 13, 14, 15, or 16.

18. A method of determining one or more multicomponent chemical compositions comprising:
conducting a plurality of experiments using a high-throughput automated experimentation apparatus;
for each experiment, electronically storing:
a set of experimental parameters;
a set of experimental results;
a set of molecular descriptors characterizing an aspect of the experiment;
associating data representing each experiment with previously stored data by querying a database comprising information not derived from the plurality of experiments;
processing at least a portion of the experiment data and the associated previously stored data with a processor programmed to apply a discriminator algorithm to associate at east one experiment with at least one classification;
determining one or more multicomponent chemical compositions based on the at east one classification.

19. The method of claim 18, wherein the discriminator algorithm comprises a predictive model.

20. The method of claim 19, wherein the predictive model further comprises a Kohonen neural network.

21. A method of determining one or more solid forms of a compound comprising:
conducting a plurality of experiments using a high-throughput automated experimentation apparatus;
for each experiment, electronically storing:
a set of experimental parameters;
a set of experimental results;
a set of molecular descriptors characterizing an aspect of the experiment;
associating data representing each experiment with previously stored data by querying a database comprising information not derived from the plurality of experiments;
processing at least a portion of the experiment data and the associated previously stored data with a processor programmed to apply a discriminator algorithm to associate at least one experiment with at least one classification;
determining one or more solid forms based on the at least one classification.

22. The method of claim 21, wherein the discriminator algorithm comprises a predictive model.

23. The method of claim 22, wherein the predictive model further comprises a Kohonen neural network.

24. A system for determining a multicomponent chemical composition comprising:
a database comprising at least one table, the at least one table further comprising:
a plurality of molecular descriptors;
a plurality of compound identifiers;
a plurality of compound/descriptor relations associating at least a portion of the compound identifiers with molecular descriptors;
a plurality of empirically determined physical, chemical and biological parameters;
a plurality of compound/parameter relations associating one or more compound identifiers with one or more of the empirically determined physical, chemical and biological parameters;
data representing results from a plurality of experiments performed with a
high-throughput automated experimentation apparatus;
a query system for selecting subsets of related information from the at least one table;
a multidimensional representation generation module capable of generating visual representations of data sets having at least four dimensions;
a plurality of modeling modules, each module capable of receiving information selected by the query system and estimating at least one property of a formulation.

25. A system for determining a solid form of a compound comprising:
a database comprising at least one table, the at least one table further comprising:
a plurality of molecular descriptors;
a plurality of compound identifiers;
a plurality of compound/descriptor relations associating compound identifiers with molecular descriptors;
a plurality of empirically determined physical, chemical and biological parameters;
a plurality of compound/parameter relations associating one or more compound identifiers with one or more of the empirically determined physical, chemical and biological parameters;
data representing results from a plurality of experiments performed with a
high-throughput automated experimentation apparatus;
a query system for selecting subsets of related information from the at least one table;
a multidimensional representation generation module capable of generating visual representations of data sets having at least four dimensions;
a plurality of modeling modules, each module capable of receiving information selected by the query system and estimating at least one property of a formulation.

26. The method of claim 24 or 25 wherein the molecular descriptors comprise two or more of the following:
the number of oxygen atoms;
the number of nitrogen atoms;
the number of free carboxylic acid groups;
the number of free primary amine groups;
the number of secondary amine groups;
the total number of amine groups;
the number of hydroxyl groups;
the number of methyl groups;
the number of amide groups;
the number of acid halide groups;
the number of aldehyde groups;
the number of amine oxide groups;
the number of benzene rings;
the number of azo groups;
the number of epoxy rings;
the number of cyclohexyl rings;
the number of isocyanate moities;
the number of ketone moities;
the number of isopropyl groups
the number of ethyl groups;
the number of propyl groups;
the number of butyl groups;
the number of pentyl groups;
the number of hexyl groups;
the number of heptyl groups;
the number of octyl groups;
the number of nonyl groups;
the number of glucose moities;
the number of sucrose moieties;
the number of fructose moities;
the number of amino acids;
the number of peptides;
the molal volume;
the diffusivity;
the molecular weight;
the number of carbon atoms;
the number of halogen atoms;
the total number of N and O atoms;
the proximity effect of N and O;
the number of unsaturated bonds;
the number of aromatic polar substituents;
the critical micelle concentration;
dissociation constant;
partition coefficient;
interatomic distance;
unit cell dimension;
unit cell angle;
pH;
van der Waals radius;
partial charge;
melting point;
boiling point;
sublimation point;
glass transition temperature;
dipole moment;
force constant;
torsional barrier;
inversion barrier;
bond strength;
bond angle;
quantum yield;
delocalization energy;
resonance energy;
compression energy;
molarity;
molality;
density;
viscosity;
dielectric constant;
refractive index;
vapor pressure;
transition energy;
solvent ionizing power;
solubility parameter;
water solubility;
thermal conductivity;
electrical conductivity;
pKa;
atomic radius;
valence;
electronegativity;
electron affinity;
ionization potential;
atomic weight;
atomic number;
stretching force constant;
bending force constant;
volume of activation;
Hammett substituent constant;
chemical shift;
polarizability factor;
NMR coupling constant;
absorbance;
transmittance;
optical purity;
specific rotation;
mole fraction;
mass-to-charge ratio;
charge density.

27. The system of claim 24 or 25, wherein the biological parameters comprise an indicator of one or more of:
permeability of a mammal's gastrointestinal membrane;
bioavailability;
taste;
toxicity;
metabolic profile;
color;
smell;
potency;
therapeutic effect.

28. A method for producing crystals comprising:
(a) electronically calculating a set of predicted crystal polymorphs of a target compound;
(b) electronically calculating expected experimental results for at least a portion of the predicted polymorphs;
(c) conducting a first plurality of crystallization experiments using a high-throughput automated experimentation apparatus;
(d) electronically comparing at least a portion of the expected experimental results with the actual experimental results to determine which of the at least a portion of the predicted polymorphs were produced.

29. The method of claim 28 wherein the crystal structure of the target compound is predicted using hydrogen-bond-biased simulated annealing.

30. A method for determining a solid form of a compound comprising:
(a) predicting a crystal structure of a target chemical species;
(b) selecting a first range of conditions for crystal generation;
(c) conducting a first plurality of experiments within the first range of conditions using a high-throughput automated experimentation apparatus;
(d) testing at least a portion of the experimental results for the presence of crystals;
(e) classifying at least a portion of the experiments based on predicted crystal forms;
(f) selecting a second range of conditions for crystal generation based on one or more of the classifications;
(g) conducting a second plurality of experiments within the second range of conditions using the high-throughput automated experimentation apparatus.

31. The method of claim 30 wherein the second range of conditions is determined based on conditions that produced desired crystals.

32. A method for preparing a crystal comprising:
(a) performing simulated hydrogen-bond-biased simulated annealing to predict a plurality of polymorphs of a target compound;
(b) calculating expected properties of at least a portion of the predicted polymorphs;
(c) conducting a plurality of crystallization experiments using a high-throughput automated experimentation apparatus;
(e) comparing measured properties of crystals produced by the plurality of crystallization experiments with the expected properties of at least a portion of the predicted polymorphs to determine which of the at least a portion of the predicted polymorphs were produced by the experiments;
(f) generating a predictive model of the relationship between experimental parameters and polymorphs produced;
(g) calculating a set of experimental parameters for a second set of crystallization experiments from the predictive model.

33. The method of claim 1, 11, 13, 18, or 24 wherein the multicomponent chemical composition comprises one or more of: a pharmaceutical, a nutraceutical, a dietary supplement, an alternative medicine, a sensory material, an agrochemical, a consumer product formulation, an industrial product formulation.

34. The method of claim 2, 12, 14, 21, 25, 30 wherein the compound comprises one or more of: a pharmaceutical, a nutraceutical, a dietary supplement, an alternative medicine, a sensory material, an agrochemical, a consumer product formulation, an industrial product formulation.

35. The method of claim 1 or 2 wherein the first collection of experimental results is determined using at least one of the techniques selected from the group consisting of mass spectroscopy, HPLC, UV Spectroscopy, fluorescence spectroscopy, gas chromatography, optical density, colorimetry
